(19) United States
(12) Patent Application Publication
(10) Pub. No.: US 2009/0214460 A9
(43) Pub. Date: Aug. 27, 2009

(54) Composition Comprising a Monomer Compound Exhibiting an Optical Property, Method Making Use of Said Composition, a Monomer Compound and a Polymer Containing Said Monomer Compound and the Use Thereof

(76) Inventor: Timo Luukas, Massy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/593,970

(22) PCT Filed: Feb. 28, 2005
(86) PCT No.: PCT/FR2005/000476

§ 371 Date: Sep. 22, 2006

Related U.S. Application Data
(60) Provisional application No. 60/560,017, filed on Apr. 7, 2004.
(60) Provisional application No. 60/560,018, filed on Apr. 7, 2004.

(30) Foreign Application Priority Data
Mar. 25, 2004 (FR) 0403083
Mar. 25, 2004 (FR) 0403085

(51) Int. Cl. A61K 31/787; A61Q 1/02; A61P 17/00

(52) U.S. Cl. 424/78.03; 424/78.36

(57) ABSTRACT

The invention relates to a cosmetic or pharmaceutical composition comprising, in a physiologically acceptable medium, at least one type of polymer containing at least one type of novel monomer compound exhibiting optical properties. Said invention also relates to a cosmetic method for making up or treating keratinous materials, in particular a body or face skin, lips, nails, eyebrows, eyelashes and/or hairs, including the application of said cosmetic composition to said materials. Novel monomer compounds exhibiting optical properties, polymers containing said compounds and the use thereof in a composition in order to transfer the optical properties thereto, in particular fluorescent or optical brightener properties are also disclosed.

### CROSS REFERENCE TO RELATED APPLICATIONS

This application is a National Phase application based on PCT/FR2005/000476, filed on Feb. 28, 2005, and claims the priority of French Application No. 0403083, filed Mar. 25, 2004, and French Application No. 0403085, filed Mar. 25, 2004, and the benefit of U.S. Provisional Application No. 60/560,017, filed on Apr. 7, 2004, and U.S. Provisional Application No. 60/560,017, filed on Apr. 7, 2004, the content of all of which is incorporated herein by reference.

The present invention relates to novel cosmetic or pharmaceutical compositions, especially for topical application, and especially to novel makeup compositions, comprising organic polymers with particular optical properties and especially fluorescence properties. The present invention also relates to novel monomeric compounds with optical properties, especially fluorescence properties, and also to polymers that may be prepared from these compounds.

Cosmetic compositions, and especially makeup compositions such as loose or compact powders, foundations, makeup rouges, eyeshadows, lipsticks or nail varnishes, generally consist of a suitable vehicle and one or more coloring agents intended to give said compositions a certain color before and/or after applying them to the skin, mucous membranes, semimucous membranes and/or the integuments such as the nails, the eyelashes or the hair.

To create colors, a fairly limited range of coloring agents is used at the present time, especially comprising lakes, mineral pigments, organic pigments and nacreous pigments.

The pigments and lakes used in the makeup field are of very diverse origin and chemical nature. Their physico-chemical properties, especially their granulometry, specific surface area, density, etc., are thus very different. These differences are reflected by variations in behavior: their ease of use or of dispersion in the medium; their light and heat stability; their mechanical properties. Mineral pigments, in particular mineral oxides, are, on the other hand, very stable to light and to pH, but give rather dull, pale colors. It is thus necessary to introduce a large amount of them into cosmetic formulations in order to obtain a sufficiently saturated mark. This high percentage of mineral particles can, however, affect the gloss of the composition. As regards nacreous pigments, they can produce varied colors, but of relatively weak intensity, which lead to iridescent effects that are usually quite weak. In the field of temporary or short-term hair dyeing, which gives rise to a slight change in the natural color of the hair that holds from one shampoo wash to the next and that serves to enhance or correct an already-established shade, coloration with common pigments to give the hair a temporary tint has already been proposed, but the shades obtained by this coloration remain quite dull, too uniform and rather boring.

In the field of makeup, only organic lakes have made it possible until now to obtain bright, vivid colors.

However, most organic lakes have very poor lightfastness, which is reflected by pronounced attenuation of their color over time. They may also be heat- and/or pH-unstable. Furthermore, certain lakes produce excessive bleeding, i.e. they have the drawback of staining the support onto which they are applied.

Thus, this may have the consequence of staining ocular lenses in the case of eyeliners or mascaras, or of leaving a coloration on the skin or the nails after makeup removal in the case of lipsticks or nail varnishes. Finally, the instability of lakes is also worsened when they are combined with photoreactive pigments, for instance titanium dioxide. Now, as it happens, these pigments are very widely used in makeup, especially for protecting against UV radiation.

Consequently, the use of organic lakes in cosmetics is quite limited, which has the consequence of limiting the shades that may be produced.

Thus, there is still a need for organic polymers with optical properties, which can be used in cosmetics, to give compositions comprising them and/or makeup obtained using these compositions adequate optical effects, said polymers moreover having good heat and photochemical stability, while at the same time producing little bleeding.

After considerable research, the Applicant has demonstrated that the use of a specific family of polymers, in fact comprising at least one specific monomer, unexpectedly allows such a result to be obtained.

Thus, one subject of the invention is a cosmetic or pharmaceutical composition comprising, in a physiologically acceptable medium, at least one polymer comprising at least one monomeric compound as defined below.

Another subject of the invention is a cosmetic process for making up or caring for keratin materials, especially bodily or facial skin, the lips, the nails, the eyelashes, the eyebrows and/or the hair, comprising the application of such a cosmetic composition to said materials.

The polymers according to the invention may be in solid or liquid form and give noteworthy optical effects to the compositions comprising them and also to the applied makeup; in particular, they can give lightening or color effects.

These optical effects may be advantageously modified as a function of the chemical nature and/or the position of the various substituents present on the monomer with an optical effect used to form the polymer. In general, when the group X is an oxygen, the resulting monomer will rather be of blue/white color; when the group X comprises a nitrogen atom, the resulting monomer will rather be in the orange range.

Among the other advantages that the polymers according to the invention can afford, mention may be made of their good heat, pH and light stability.

It has also been found that the polymers according to the invention show good solubility in fatty substances, which can vary and be adjusted according to the nature of the monomers. This good liposolubility can also facilitate their subsequent use, especially in cosmetic compositions generally comprising a fatty phase.

Furthermore, the good cosmetic properties of the compositions according to the invention are maintained when they comprise the polymers according to the invention.

Furthermore, although being of similar chemical structure, the polymers according to the invention may show, depending on the nature of the substituents, a wide variety of optical effects, which may range from blue/violet to orange/red, passing through yellow. This gives access to a range of compounds, belonging to the same chemical family, and thus being formulated in a similar manner, which offer noteworthy diversities of colors or of optical properties; this especially facilitates the task of formulators by allowing them to keep a common architecture for all of their compositions, irrespective of the polymers with optical properties used.

Moreover, we have found that the monomers according to the invention and the polymers comprising them have good fluorescence properties, and some of them have good optical-brightening properties. It is recalled that optical brighteners are endowed with fluorescence properties; in general, fluorescent compounds absorb in the ultraviolet and visible range, and re-emit energy by fluorescence at a wavelength of between 380 nm and 830 nm; when this wavelength is between 380 nm and 480 nm, i.e. in the blue region of the visible range, the compounds are then optical brighteners.

In addition, the polymers according to the invention have the advantage of undergoing makeup removal easily.

The composition according to the invention thus comprises, in a physiologically acceptable medium and especially a cosmetically or pharmaceutically acceptable medium, at least one polymer that can be obtained by polymerization, especially free-radical polymerization, of at least one monomer of formula (I).

Said monomer of formula (I) thus corresponds to the following formula:

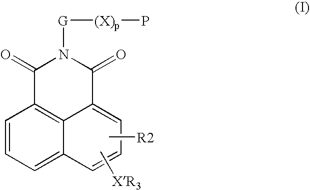

in which:
the groups R₂ and X′R₃ are present on the same ring or each on a different ring;
R₂ and R₃ represent, independently of each other, a hydrogen atom, a halogen or a linear, branched and/or cyclic, saturated and/or unsaturated carbon-based radical containing 1 to 30 carbon atoms; optionally substituted with one or more groups chosen from ═O, OH, NH₂ and halogen atoms; and/or optionally interrupted with one or more heteroatoms chosen from O, N, P, Si and S;
X and X′ represent, independently of one another, —O—, —S—, —SO—, —SO₂—, —NH— and —NR₄— with R₄ representing a linear, branched and/or cyclic, saturated and/or unsaturated carbon-based radical containing 1 to 30 carbon atoms, optionally substituted with one or more groups chosen from ═O, OH, NH₂ and halogen atoms; and/or optionally interrupted with one or more heteroatoms chosen from O, N, P, Si and S;
p is equal to 0 or 1
G is a linear, branched and/or cyclic, saturated and/or unsaturated divalent carbon-based radical containing 1 to 30 carbon atoms, optionally substituted with one or more groups chosen from ═O, OH, NH₂ and halogen atoms; and/or optionally interrupted with one or more heteroatoms chosen from O, N, P, Si and S;
P is a polymerizable group chosen from one of the following formulae:

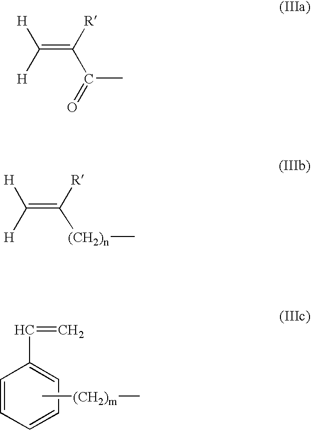

in which:
R′ represents H or a linear or branched, saturated C₁₋₆ hydrocarbon-based radical,
n is equal to 0 or 1 and m is equal to 0 or 1.

In the present invention, the term “cyclic radical” means a monocyclic or polycyclic radical, which is thus itself in the form of one or more saturated and/or unsaturated, optionally substituted rings (for example cyclohexyl, cyclodecyl, benzyl or fluorenyl), but also a radical that comprises one or more of said rings (for example p-tert-butylcyclohexyl or 4-hydroxybenzyl).

In the present invention, the term “saturated and/or unsaturated radical” means totally saturated radicals, totally unsaturated radicals, including aromatic radicals, and also radicals comprising one or more double and/or triple bonds, the rest of the bonds being single bonds.

The radical R2 is preferably a hydrogen atom.

The radical R3 is preferably a cyclic, linear and/or branched, saturated and/or unsaturated carbon-based and especially hydrocarbon-based radical, optionally comprising a hydrocarbon-based ring that is itself saturated and/or unsaturated, containing 2 to 18, especially 3 to 14 or even 6 to 12 carbon atoms, and may comprise at least one heteroatom, especially one, two or three nitrogen, sulfur and/or oxygen atoms. R3 may especially be an n-propyl, isopropyl, n-butyl, isobutyl, tert-butyl, n-pentyl, cyclopentyl, n-hexyl, cyclohexyl, n-heptyl, n-octyl, cyclooctyl, decyl, cyclodecyl, dodecyl, cyclododecyl, p-tert-butylcyclo-hexyl, benzyl or phenyl radical.

X′R3 may especially be a radical —NH—(CH₂)ₙH; —O—(CH₂)ₙH, for example ethoxy or methoxy; —S—(CH₂)ₙH, —SO—(CH₂)ₙH or —SO₂—(CH₂)ₙH with n being an integer between 1 and 30 and especially between 4 and 12; or alternatively C6-C18-NH-cycloalkyl, especially —NH-cyclohexyl, —NH-cyclooctyl, —NH-cyclodecyl or —NH-cyclododecyl; or alternatively C6-C18-S-cycloalkyl, C6-C18-SO-cycloalkyl or C6-C18-SO₂-cycloalkyl; or alternatively a radical chosen from the following:

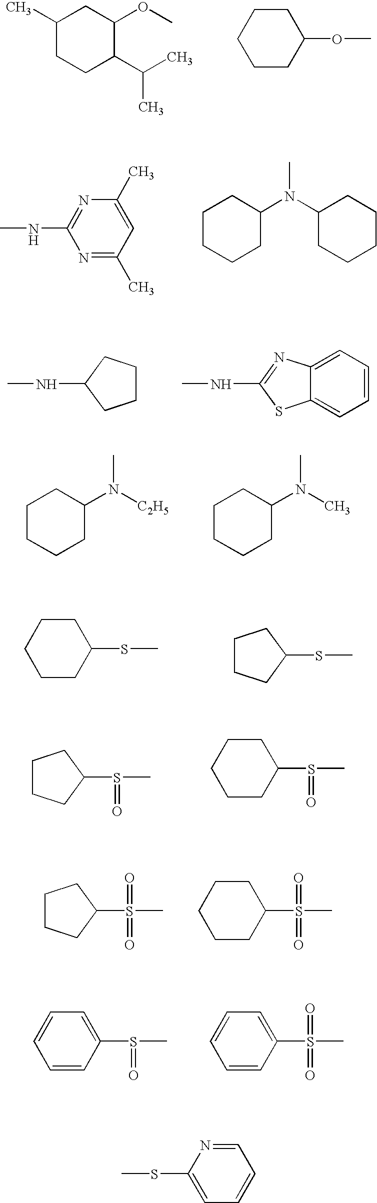

The divalent radical G is preferably a linear, branched and/or cyclic, saturated or unsaturated divalent hydrocarbon-based radical optionally comprising a hydrocarbon-based ring that is itself saturated or unsaturated, containing in total 2 to 18 and especially 3 to 8 carbon atoms, optionally substituted with one or more groups chosen from ═O, OH, NH₂ and halogen atoms; and/or optionally interrupted with one or more heteroatoms chosen from O, N, P, S and Si.

Preferentially, G is chosen from linear or branched, saturated divalent hydrocarbon-based radicals optionally comprising a saturated hydrocarbon-based ring, containing in total 2 to 16 and especially 3 to 10 carbon atoms.

Thus, G may be chosen from methylene, ethylene, n-propylene, isopropylene (or 1-methylethylene and 2-methylethylene), n-butylene, isobutylene, pentylene, especially n-pentylene, hexylene, especially n-hexylene or cyclohexylene, heptylene, octylene, cyclooctylene, decylene, cyclodecylene, cyclohexyldimethylene dodecylene, cyclododecylene.

The divalent radical X is preferably chosen from —O—, —S—, —NH— and —NR₄—, preferably O.

When it is present, the radical R4 preferentially represents a linear, branched and/or cyclic, saturated or unsaturated hydrocarbon-based radical containing 2 to 12 carbon atoms, optionally substituted with one or more groups chosen from ═O, OH and NH₂. R4 may especially be an ethyl, n-propyl, isopropyl, n-butyl, isobutyl, tert-butyl, pentyl, hexyl, cyclohexyl, octyl, decyl, dodecyl, phenyl or benzyl radical.

Preferably, p is equal to 1.

The polymerizable group P is preferably chosen from one of the following formulae:

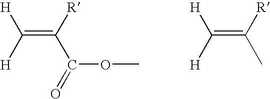

in which R′ represents H or methyl.

Among the monomeric compounds that are particularly preferred according to the invention, mention may be made of the compounds corresponding to one of the following formulae:

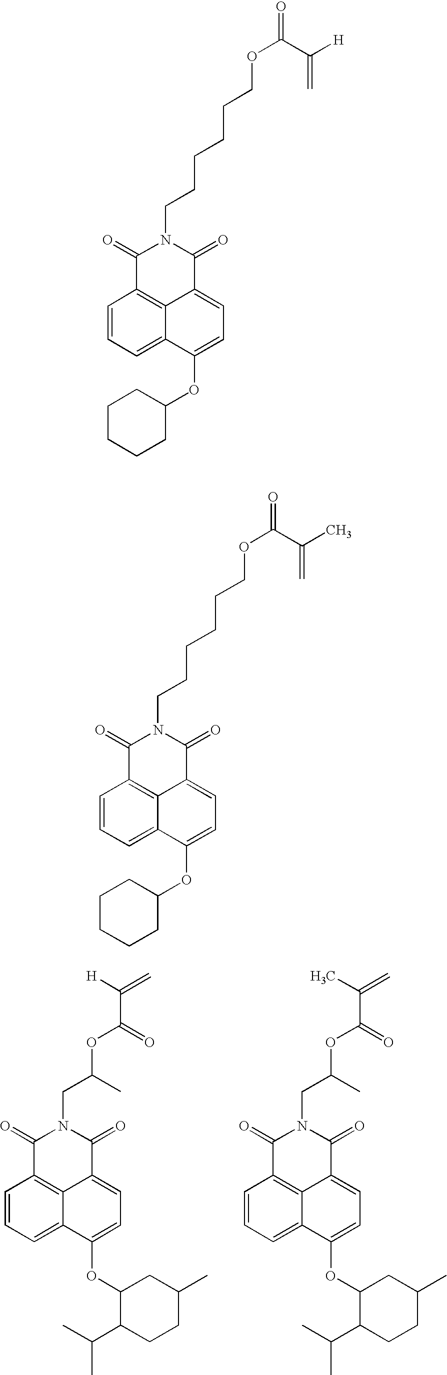

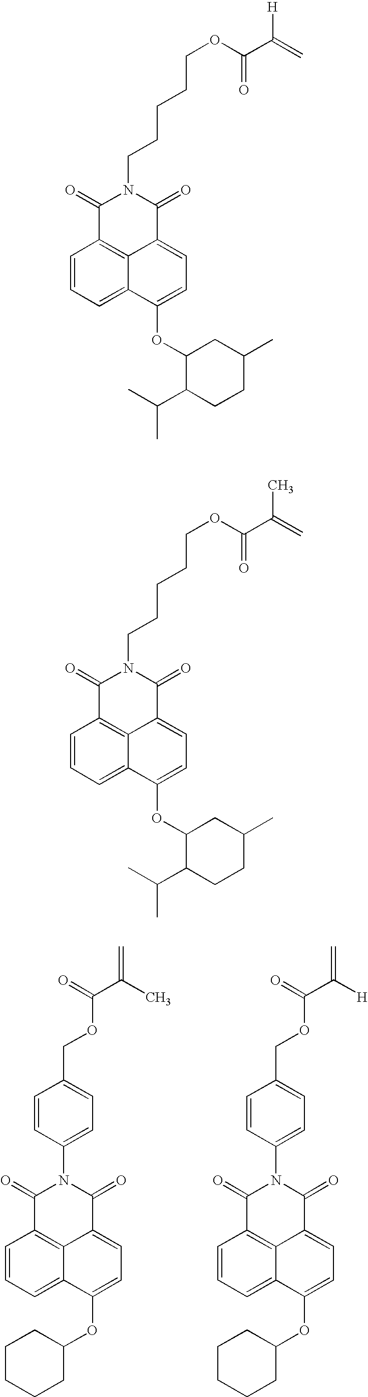

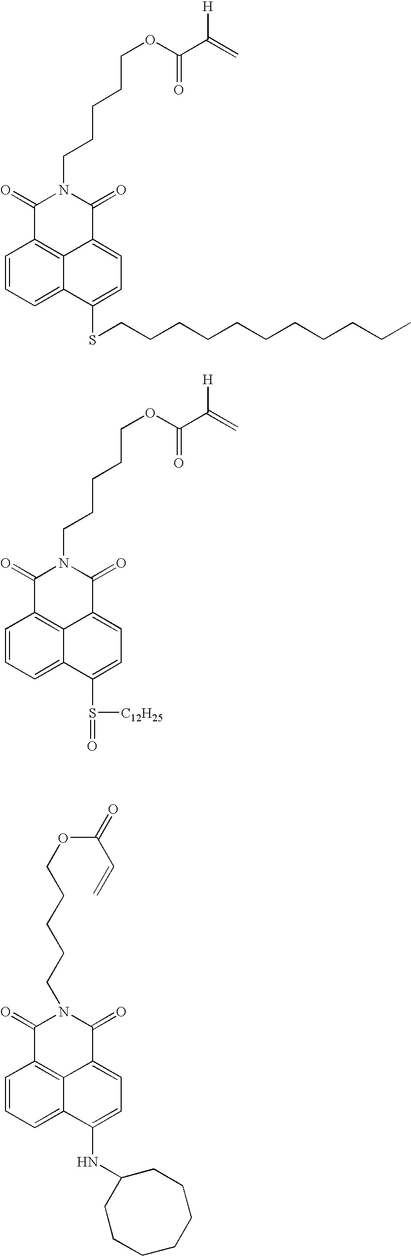

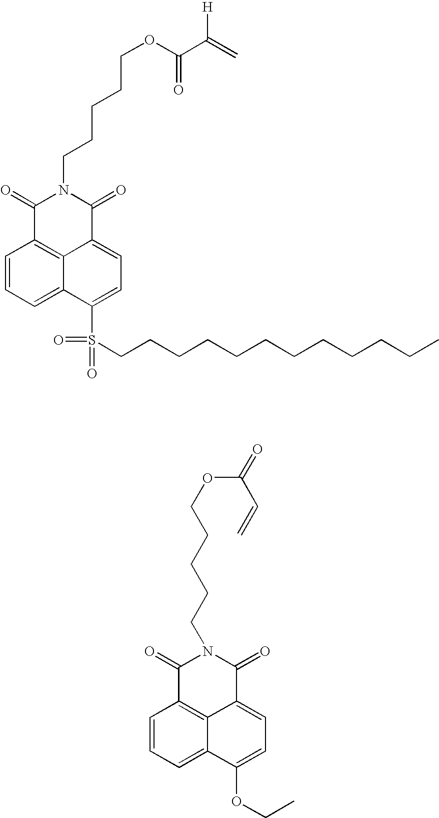

Another subject of the present invention is a monomeric compound of formula (I) below:

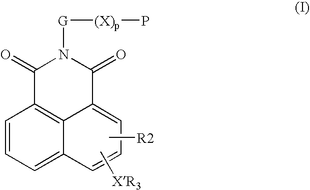

in which:
in which:
the groups R2 and X′R3 are present on the same ring or each on a different ring;
R2 and R3 represent, independently of each other, a hydrogen atom, a halogen or a linear, branched and/or cyclic, saturated and/or unsaturated carbon-based radical, containing 1 to 30 carbon atoms; optionally substituted with one or more groups chosen from ═O, OH, NH₂ and halogen atoms; and/or optionally interrupted with one or more heteroatoms chosen from O, N, P, Si and S;
X and X′ represent, independently of one another, —O—, —S—, —SO—, —SO₂—, —NH— and —NR₄— groups with R₄ representing a linear, branched and/or cyclic, saturated and/or unsaturated carbon-based radical containing 2 to 30 carbon atoms, optionally substituted with one or more groups chosen from ═O, OH, NH₂ and halogen atoms; and/or optionally interrupted with one or more heteroatoms chosen from O, N, P, Si and S;
p is equal to 0 or 1,
G is a linear, branched and/or cyclic, saturated and/or unsaturated divalent carbon-based radical, containing 1 to 30 carbon atoms, optionally substituted with one or more groups chosen from ═O, OH, NH₂ and halogen atoms; and/or optionally interrupted with one or more heteroatoms chosen from O, N, P, Si and S;
P is a polymerizable group chosen from one of the following formulae:

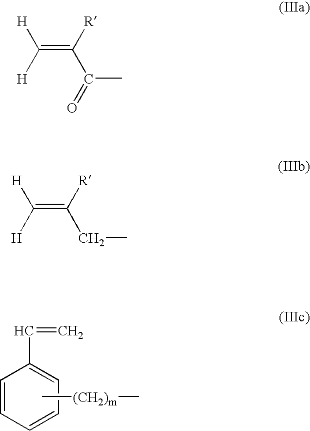

in which:
R′ represents H or a linear or branched, saturated C₁₋₆ hydrocarbon-based radical,
m is equal to 0 or 1;
   given that:
when R2=H and simultaneously P is of formula (IIIb), then X′R3 is other than OCH₃;
when R2=H and simultaneously P is of formula (IIIa), and X is equal to O, NH or NR₄, and X′ is equal to O, S or NR₄, then R3 is chosen from (i) optionally substituted and/or optionally interrupted saturated, linear or branched C2-C5 or C7-C24 alkyl radicals and (ii) optionally substituted and/or optionally interrupted cyclic C5-C18 saturated alkyl radicals.

The monomeric compounds of formula (I) in which:
R2 is hydrogen, X′ is O, NH or NR₄, and R3 is a linear, branched and/or cyclic, saturated and/or unsaturated carbon-based radical containing 2 to 12 carbon atoms; and/or
G is a linear, branched and/or cyclic, saturated and/or unsaturated divalent carbon-based radical containing 1 to 8 carbon atoms, are most particularly preferred.

Another subject of the invention is a polymer comprising at least one such monomeric compound.

Another subject of the invention is the use of such a monomeric compound or of such a polymer comprising it, in a composition, for giving said composition optical effects, especially fluorescence or optical-brightening effects.

The novel monomers, and the polymers comprising them, have good optical properties and can be prepared more easily than those of the prior art.

For the purpose especially of industrial exploitation, monomeric and polymeric compounds of high reactivity are sought, which allows a short reaction (polymerization) time.

Monomers and polymers that have good optical properties, with a wide color range, and which can be used in cosmetics are also sought.

It is found that with the compounds according to the present invention, the polymerization is easier, especially on account of the presence of a spacer group (G).

In addition, the polymers and the monomeric compounds according to the invention find a most particular use for giving a composition optical effects, especially fluorescence or optical-brightening effects.

Some of these compounds may especially be prepared according to the prior art, for example according to the teaching of document EP 728 745, in particular the compounds for which X is N.

Schematically, the general synthetic process, may be represented as follows:

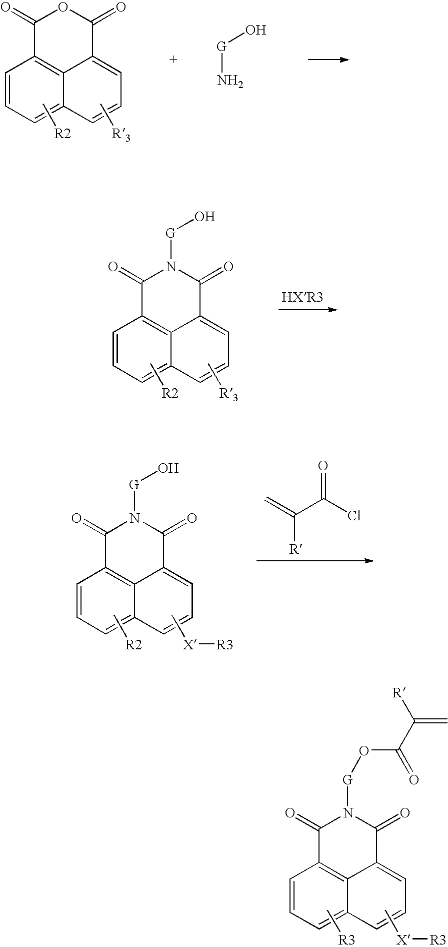

The appropriate naphthalic anhydride may thus be reacted with an appropriate primary amine HO-G-NH₂.

Preferably, the naphthalic amine is present in slight excess relative to the naphthalic anhydride, especially in a proportion of from 1 to 1.5 equivalents and preferably 1.1 equivalents per 1 equivalent of anhydride.

The reaction may be performed in a solvent chosen from solvents in which the anhydride is soluble, and especially toluene, xylene, acetic acid, NMP or ethanol; the reaction is preferably performed at the reflux temperature of the solvent, for example at a temperature of 50-250° C. and preferably 75-150° C.

The imide formed can then be reacted with an alcohol or a thiol.

For example, when R′₃ is a halogen (preferably chlorine or bromine), it is possible to perform an aromatic nucleophilic substitution, for example using an alcohol or a thiol, such as ethanol, cyclohexanol or dodecanethiol.

The reaction may be performed in the absence of solvent, or in the presence of a dipolar aprotic solvent such as dichloromethane or THF (tetrahydro-furan), especially at a temperature of 20-150° C.

The sulfur derivatives may be oxidized under mild conditions so as to give the corresponding sulfoxide. By modifying the oxidation conditions, it is also possible to prepare the corresponding sulfone. These sulfides, sulfoxides and sulfones may then be converted in order to obtain the desired methacrylates or acrylates.

As regards the alcohol derivatives, they may be reacted with a (meth)acryloyl halide, especially a chloride, so as to form the corresponding (meth)acrylate.

This reaction may be performed in the presence of a base such as triethanolamine, in a solvent such as tetrahydrofuran or dichloromethane, especially at a temperature of from −30° C. to 100° C. and preferably from 0 to 80° C.

These monomeric compounds may be used as first monomer to prepare copolymers comprising them.

In particular, the monomeric compounds with an optical effect according to the invention may be used to prepare homopolymers or copolymers comprising only monomeric compounds with an optical effect, of formula (I), alone or as a mixture, or alternatively of formula (I) as a mixture with others, especially as a mixture with those of formula A, B and/or C as defined below, these various compounds then each possibly being present, for example, in a proportion of from 0.5% to 99.5% by weight, especially 5% to 95% by weight or even 10% to 90% by weight, and better still each in a proportion of 30% to 70% by weight relative to the total weight of the polymer. This may especially make it possible to prepare polymers with a wide range of optical effects (especially color, optical brightening or the like).

Among the monomeric compounds with an optical effect that may be copolymerized with the monomeric compounds of formula (I), and optionally with one or more of the additional comonomers as defined below, mention may be made of the compounds of formula (A), (B) and/or (C):

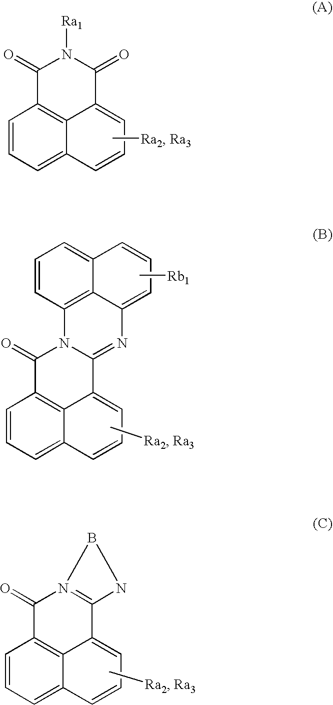

in which:
Ra1 represents a linear, branched and/or cyclic, saturated and/or unsaturated carbon-based radical containing 1 to 32 carbon atoms; optionally substituted with one or more groups chosen from ═O, OH, NH₂ and halogen atoms; and/or optionally interrupted with one or more heteroatoms chosen from O, N, P, Si and S;
Rb1 is chosen from (i) a hydrogen atom, (ii) a halogen, (iii) a linear, branched and/or cyclic, saturated and/or unsaturated carbon-based radical containing 1 to 12 carbon atoms, optionally substituted with one or more groups chosen from ═O, OH and NH₂ and/or optionally interrupted with one or more heteroatoms chosen from O, N, P, Si and S; (iv) a group NRR′ with R and R′ being, independently of each other, a hydrogen atom or a linear, cyclic or branched, saturated C1-6 hydrocarbon-based radical, especially methyl, ethyl, propyl, isopropyl, n-butyl, isobutyl, tert-butyl, pentyl or hexyl;
Ra2 and Ra3, which are present on the same ring or each on a different ring, represent, independently of each other, a hydrogen, a halogen or a group of formula -Xa-Ga—Pa (II), with the proviso that at least one of the radicals Ra2 and/or Ra3 represents a group of formula (II), in which:
Xa is chosen from the groups —O—, —S—, —SO—, —SO₂—, —NH— and —NR₄— with R4 representing a linear, branched and/or cyclic, saturated and/or unsaturated carbon-based radical containing 1 to 30 carbon atoms, optionally substituted with one or more groups chosen from ═O, OH, NH₂ and halogen atoms; and/or optionally interrupted with one or more heteroatoms chosen from O, N, P, Si and S;
Ga is a linear, branched and/or cyclic, saturated and/or unsaturated divalent carbon-based radical containing 1 to 32 carbon atoms, optionally substituted with one or more groups chosen from ═O, OH, NH₂ and halogen atoms; and/or optionally interrupted with one or more heteroatoms chosen from O, N, P, Si and S;
Pa is a polymerizable group chosen from one of the following formulae:

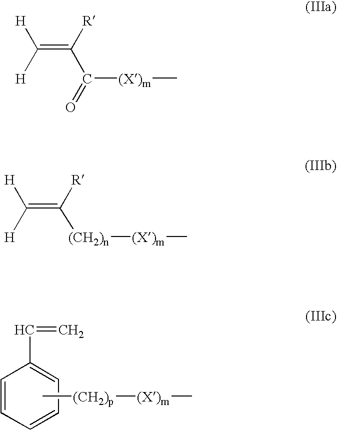

in which:
R′ represents H or a linear or branched, saturated C₁-6 hydrocarbon-based radical,
X′ represents O, NH or NR″ with R″ representing a radical chosen from C1-6 alkyl, C6-10 aryl, (C6-10)aryl(C1-6)alkyl and (C1-6)alkyl(C6-10)aryl radicals, the alkyl and/or aryl groups also possibly being substituted with one or more groups chosen from OH, halogen, C1-6 alkoxy and C6-10 aryloxy; and
m is equal to 0 or 1; n is equal to 0 or 1; p is equal to 0, 1 or 2;
B represents one of the following divalent aromatic groups (IVa) to (IVd):

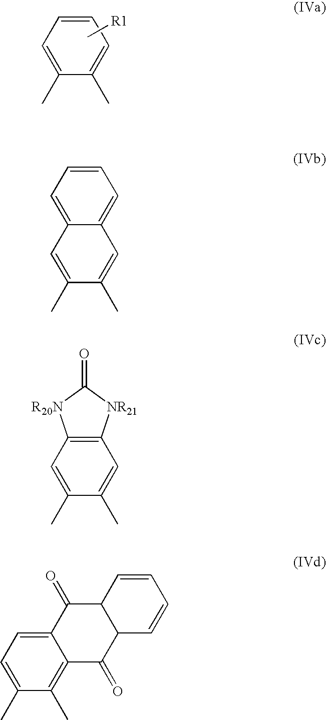

in which:
R1 is a linear, branched and/or cyclic, saturated and/or unsaturated carbon-based radical containing 1 to 32 carbon atoms, optionally substituted with one or more groups chosen from ═O, OH, NH₂ and halogen atoms;
R20 and R21 are, independently of each other, a hydrogen atom, a linear or branched C1-8 alkyl radical or a cyclopentyl, cyclohexyl, cyclooctyl, cyclodecyl, cyclododecyl, benzyl, naphthyl or phenyl radical.

The copolymers according to the invention may be statistical, alternating, grafted or block, for example diblock or triblock, copolymers comprising said monomeric compounds with an optical effect according to the invention and additional comonomers.

The monomeric compounds according to the invention may form all or part of a block, or even several blocks. Block copolymers of the type A-B, ABA, BAB or ABC in which A is a block comprising the monomeric compound(s) according to the invention may thus be prepared, optionally as a mixture with additional comonomers, B and C being different blocks, comprising additional comonomers, alone or as a mixture, and identical to or different than the comonomers present in the block A.

The copolymers comprising the monomeric compounds according to the invention may also be of the gradient type.

In these copolymers, the monomeric compounds with an optical effect may be present in an amount of from 0.01% to 70% by weight relative to the weight of the final polymer, especially in an amount of from 0.1% to 50% by weight, in particular from 0.5% to 30% by weight or even from 1% to 20% by weight and better still from 2% to 10% by weight, the additional comonomers, alone or as a mixture, representing the remainder to 100% by weight.

The copolymers according to the invention may comprise, in addition to the monomeric compound(s) with an optical effect, at least one additional comonomer that is hydrophilic, or a mixture of such comonomers.

These hydrophilic comonomers may be present in a proportion of from 1% to 99.99% by weight, especially 2-70% by weight, better still 5-50% by weight or even 10-30% by weight, relative to the total weight of the copolymer.

In the present description, the term “hydrophilic monomer” will denote, without preference, monomers whose homopolymers are soluble or dispersible in water, or of which one ionic form is soluble or dispersible in water.

A homopolymer is said to be water-soluble if it forms a clear solution when it is in solution at 5% by weight in water, at 25° C.

A homopolymer is said to be water-dispersible if, at 5% by weight in water, at 25° C., it forms a stable suspension of fine, generally spherical particles. The mean size of the particles constituting said dispersion is less than 1 μm and more generally ranges between 5 and 400 nm and preferably from 10 to 250 nm. These particle sizes are measured by light scattering.

A monomer will be said to be “hydrophobic” if it is not hydrophilic.

Preferably, the additional hydrophilic comonomer(s) has a Tg of greater than or equal to 20° C. and especially greater than or equal to 50° C., but may optionally have a Tg of less than or equal to 20° C.

The copolymers according to the invention may comprise at least one additional hydrophobic comonomer, or a mixture of such comonomers.

These additional hydrophobic comonomers may be present in a proportion of from 1% to 99.99% by weight, especially 30-98% by weight, better still 50-95% by weight or even 70-90% by weight relative to the total weight of the copolymer.

Preferably, the hydrophobic comonomer has a Tg of greater than or equal to 20° C. and especially greater than or equal to 30° C., but may optionally have a Tg of less than or equal to 20° C.

In the present invention, the Tg (or glass transition temperature) is measured according to ASTM standard D3418-97, by differential thermal analysis (DSC “Differential Scanning Calorimetry”) on a calorimeter, over a temperature range of between −100° C. and +150° C. at a heating rate of 10° C./minute in 150 μl aluminum crucibles.

In general, as additional comonomer that may be copolymerized with at least one monomeric compound of formula (I), mention may be made, alone or as a mixture, of the following monomers:
(i) ethylenic hydrocarbons containing from 2 to 10 carbons, such as ethylene, isoprene or butadiene;
(ii) the (meth)acrylates of formula:

CH₂═CHCOOR′₃ or

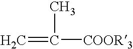

in which R′₃ represents:
a linear or branched alkyl group of 1 to 18 carbon atoms, in which is (are) optionally intercalated one or more heteroatoms chosen from O, N, S and P; said alkyl group also possibly being optionally substituted with one or more substituents chosen from hydroxyl groups, halogen atoms (Cl, Br, I and F), and groups Si(R₄R₅), in which R₄ and R₅, which may be identical or different, represent a C₁ to C₆ alkyl group or a phenyl group; R′₃ may especially be a methyl, ethyl, propyl, n-butyl, isobutyl, tert-butyl, hexyl, ethylhexyl, octyl, lauryl, isooctyl, isodecyl, dodecyl, cyclohexyl, t-butyl-cyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,
a C₃ to C₁₂ cycloalkyl group such as an isobornyl group,
a C₃ to C₂₀ aryl group such as a phenyl group,
a C₄ to C₃₀ aralkyl group (C₁ to C₈ alkyl group) such as 2-phenylethyl, t-butylbenzyl or benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
   said cycloalkyl, aryl, aralkyl, heterocyclic or heterocycloalkyl groups possibly being optionally substituted with one or more substituents chosen from hydroxyl groups, halogen atoms and linear or branched C₁₋₄ alkyl groups in which is (are) optionally intercalated one or more heteroatoms chosen from O, N, S and P, said alkyl groups also possibly being optionally substituted with one or more substituents chosen from hydroxyl groups, halogen atoms (Cl, Br, I and F), and groups Si (R₄R₅), in which R₄ and R₅, which may be identical or different, represent a C₁ to C₆ alkyl group or a phenyl group,
R′₃ may also be a group —(C₂H₄O)ₘ—R″, with m=5 to 150 and R″=H or C₁ to C₃₀ alkyl, for example —POE-methyl or —POE-behenyl;
(iii) the (meth)acrylamides of formula:

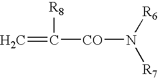

in which R₈ denotes H or methyl; and R₇ and R₆, which may be identical or different, represent:
a hydrogen atom; or
a linear or branched alkyl group of 1 to 18 carbon atoms, in which is (are) optionally intercalated one or more heteroatoms chosen from O, N, S and P; said alkyl group also possibly being optionally substituted with one or more substituents chosen from hydroxyl groups, halogen atoms, (Cl, Br, I and F), and groups Si(R₄R₅), in which R₄ and R₅, which may be identical or different, represent a C₁ to C₆ alkyl group or a phenyl group; R₆ and/or R₇ may especially be a methyl, ethyl, propyl, n-butyl, isobutyl, tert-butyl, hexyl, ethylhexyl, octyl, lauryl, isooctyl, isodecyl, dodecyl, cyclohexyl, t-butylcyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,
a C₃ to C₁₂ cycloalkyl group, such as an isobornyl group,
a C₃ to C₂₀ aryl group such as a phenyl group,
a C₄ to C₃₀ aralkyl group (C₁ to C₈ alkyl group) such as 2-phenylethyl, t-butylbenzyl or benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl, said cycloalkyl, aryl, aralkyl, heterocyclic or heterocycloalkyl groups possibly being optionally substituted with one or more substituents chosen from hydroxyl groups, halogen atoms and linear or branched C₁-C₄ alkyl groups in which is (are) optionally intercalated one or more heteroatoms chosen from O, N, S and P, said alkyl groups also possibly being optionally substituted with one or more substituents chosen from hydroxyl groups, halogen atoms (Cl, Br, I and F) and groups Si(R₄R₅), in which R₄ and R₅, which may be identical or different, represent a C₁ to C₆ alkyl group or a phenyl group.

Examples of (meth)acrylamide monomers are (meth)acrylamide, N-ethyl(meth)acrylamide, N-butyl-acrylamide, N-t-butylacrylamide, N-isopropylacrylamide, N,N-dimethyl(meth)acrylamide, N,N-dibutylacrylamide, N-octylacrylamide, N-dodecylacrylamide, undecyl-acrylamide and N(2-hydroxypropylmethacrylamide).
(iv) the vinyl compounds of formulae:

CH₂═CH—R₉, CH₂═CH—CH₂—R₉ or CH₂═C(CH₃)—CH₂—R₉

in which R₉ is a hydroxyl group, halogen (Cl or F), NH₂, OR₁₀ in which R₁₀ represents a phenyl group or a C₁ to C₁₂ alkyl group (the monomer is a vinyl or allylic ether); acetamide(NHCOCH₃); a group OCOR₁₁ in which R₁₁ represents a linear or branched alkyl group of 2 to 12 carbons (the monomer is a vinyl or allylic ester); or a group chosen from:
a linear or branched alkyl group of 1 to 18 carbon atoms, in which is (are) optionally intercalated one or more heteroatoms chosen from O, N, S and P; said alkyl group also possibly being optionally substituted with one or more substituents chosen from hydroxyl groups, halogen atoms (Cl, Br, I and F) and groups Si(R₄R₅), in which R₄ and R₅, which may be identical or different, represent a C₁ to C₆ alkyl group or a phenyl group;
a C₃ to C₁₂ cycloalkyl group such as isobornyl or cyclohexane,
a C₃ to C₂₀ aryl group such as phenyl,
a C₄ to C₃₀ aralkyl group (C₁ to C₈ alkyl group) such as 2-phenylethyl; benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl, said cycloalkyl, aryl, aralkyl, heterocyclic or heterocycloalkyl groups possibly being optionally substituted with one or more substituents chosen from hydroxyl groups, halogen atoms and linear or branched 1 to 4 C alkyl groups in which is (are) optionally intercalated one or more heteroatoms chosen from O, N, S and P, said alkyl groups also possibly being optionally substituted with one or more substituents chosen from hydroxyl groups, halogen atoms (Cl, Br, I and F) and groups Si(R₄R₅) in which R₁ and R₂, which may be identical or different, represent a C₁ to C₆ alkyl group, or a phenyl group.

Examples of vinyl monomers are vinyl cyclohexane and styrene.

Examples of vinyl esters are vinyl acetate, vinyl propionate, vinyl butyrate, vinyl ethylhexanoate, vinyl neononanoate and vinyl neododecanoate.

Among the vinyl ethers that may be mentioned are methyl vinyl ether, ethyl vinyl ether and isobutyl vinyl ether.
(v) (meth)acrylic, (meth)acrylamide or vinyl monomers containing a fluoro or perfluoro group, such as ethylperfluorooctyl or 2-ethylperfluorohexyl (meth)acrylate;
(vi) silicone-based (meth)acrylic, (meth)acrylamide or vinyl monomers, such as methacryloxypropyltris(trimethylsiloxy)silane or acryloxypropylpolydimethylsiloxane;
(vii) ethylenically unsaturated monomers comprising at least one carboxylic, phosphoric or sulfonic acid, or anhydride, function, for instance acrylic acid, methacrylic acid, crotonic acid, maleic anhydride, itaconic acid, fumaric acid, maleic acid, acrylamidopropanesulfonic acid, vinylbenzoic acid and vinylphosphoric acid, and the salts thereof;
(viii) ethylenically unsaturated monomers comprising at least one tertiary amine function, for instance 2-vinylpyridine, 4-vinylpyridine, dimethylaminoethyl methacrylate, diethylaminoethyl methacrylate and dimethylaminopropylmethacrylamide, and the salts thereof.

The salts may be formed by neutralization of the anionic groups with a mineral base, such as LiOH, NaOH, KOH, Ca(OH)₂, NH₄OH or Zn(OH)₂; or with an organic base such as a primary, secondary or tertiary alkylamine, especially triethylamine or butylamine. This primary, secondary or tertiary alkylamine may comprise one or more nitrogen and/or oxygen atoms and may thus comprise, for example, one or more alcohol functions; mention may be made especially of amino-2-methyl-2-propanol, triethanolamine and dimethylamino-2-propanol. Mention may also be made of lysine or 3-(dimethylamino)propylamine.

Mention may also be made of the salts of mineral acids, such as sulfuric acid, hydrochloric acid, hydrobromic acid, hydriodic acid, phosphoric acid or boric acid. Mention may also be made of the salts of organic acids, which may comprise one or more carboxylic, sulfonic or phosphonic acid groups. They may be linear, branched, or cyclic aliphatic acids, or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

It is obviously possible to use several of the additional comonomers mentioned above.

The additional comonomer(s) may be present in an amount of from 30% to 99.99% by weight, especially in an amount of from 50% to 99.9% by weight, in particular from 70% to 99.5% by weight, or even from 80% to 99% by weight, and better still from 90% to 98% by weight, relative to the weight of the final polymer.

The additional comonomers are more particularly chosen, alone or as a mixture, from C₁-C₁₈ alkyl or C₃-C₁₂ cycloalkyl(meth)acrylates, and especially from methyl acrylate, methyl methacrylate, isobornyl acrylate, isobornyl methacrylate, isobutyl acrylate, isobutyl methacrylate, 2-ethylhexyl acrylate, 2-ethylhexyl methacrylate, dodecyl acrylate, dodecyl methacrylate, stearyl acrylate, stearyl methacrylate, trifluoroethyl acrylate and trifluoroethyl methacrylate.

Mention may also be made of acrylic acid, methacrylic acid, methacryloxypropyltris(trimethylsiloxy)silane, acryloxypropyltris(trimethylsiloxy)silane, acryloxy-propylpolydimethylsiloxane and methacryloxypropylpoly-dimethylsiloxane.

Said polymers may be prepared according to the methods known to those skilled in the art, especially by radical polymerization; controlled radical polymerization, for example with xanthans, dithiocarbamates or dithio esters; by polymerization using precursors of nitroxide type; by atom transfer radical polymerization (ATRP); by group transfer polymerization.

The polymerization may conventionally be performed in the presence of a polymerization initiator, which may be a radical initiator, and especially which may be chosen from organic peroxide compounds such as dilauroyl peroxide, dibenzoyl peroxide, tert-butyl peroxy-2-ethylhexanoate; or from diazo compounds such as azobisisobutyronitrile or azobisdimethylvalero-nitrile. The reaction may also be initiated using photoinitiators or via radiation of UV type, with neutrons or with plasma.

The monomeric compounds with an optical effect, and also preferably the homopolymers or copolymers comprising them, preferably have an absorption wavelength of between 200 and 550 nm, especially between 220 and 520 nm or even between 240 and 500 nm.

They preferably have an emission wavelength of between 350 and 750 nm, especially between 390 and 700 nm, or even between 420 and 670 nm.

The weight-average molecular mass (Mw) of the copolymers according to the invention is preferably between 5000 and 600 000 g/mol, especially between 10 000 and 300 000 g/mol and better still between 20 000 and 150 000 g/mol.

The weight-average (Mw) and number-average (Mn) molecular masses are determined by gel permeation liquid chromatography (GPC), eluting with THF, on a calibration curve established with linear polystyrene standards, using a refractometric and UV detector.

The polymers according to the invention, whether they are homopolymers or copolymers, may be present, alone or as a mixture, in the compositions according to the invention in an amount of from 0.01% to 60% by weight, preferably 0.1% to 50% by weight, especially 1% to 25% by weight, or even 3% to 15% by weight and better still 5% to 12% by weight, relative to the total weight of the composition.

They may be present in the composition in dissolved form, for example in water, in an oil or in an organic solvent, or alternatively in the form of an aqueous or organic dispersion.

Advantageously, the polymers according to the invention are soluble or dispersible in at least one of the phases of the composition comprising them.

The cosmetic or pharmaceutical compositions according to the invention comprise, besides said polymers, a physiologically acceptable medium, especially a cosmetically, dermatologically or pharmaceutically acceptable medium, i.e. a medium that is compatible with keratin materials such as facial or bodily skin, the hair, the eyelashes, the eyebrows and the nails.

The composition may thus comprise a hydrophilic medium comprising water or a mixture of water and hydrophilic organic solvent(s), for instance alcohols and especially linear or branched lower monoalcohols containing from 2 to 5 carbon atoms, for instance ethanol, isopropanol or n-propanol, and polyols, for instance glycerol, diglycerol, propylene glycol, sorbitol or pentylene glycol, and polyethylene glycols, or alternatively hydrophilic C₂ ethers and C₂-C₄ aldehydes.

The water or the mixture of water and hydrophilic organic solvents may be present in the composition according to the invention in a content ranging from 0.1% to 99% by weight and preferably from 10% to 80% by weight relative to the total weight of the composition.

The composition may also be anhydrous.

The composition may also comprise a fatty phase which may comprise fatty substances that are liquid at room temperature (in general 25° C.) and/or fatty substances that are solid at room temperature, such as waxes, pasty fatty substances and gums, and mixtures thereof. These fatty substances may be of animal, plant, mineral or synthetic origin. This fatty phase may also contain lipophilic organic solvents.

As fatty substances that are liquid at room temperature, often referred to as oils, which may be used in the invention, mention may be made of: hydrocarbon-based oils of animal origin such as perhydrosqualene; hydrocarbon-based plant oils such as liquid triglycerides of fatty acids of 4 to 10 carbon atoms, for instance heptanoic or octanoic acid triglycerides, or alternatively sunflower oil, maize oil, soybean oil, grapeseed oil, sesame seed oil, apricot oil, macadamia oil, castor oil, avocado oil, caprylic/capric acid triglycerides, jojoba oil, shea butter, linear or branched hydrocarbons of mineral or synthetic origin, such as liquid paraffin and derivatives thereof, petroleum jelly, polydecenes, hydrogenated polyisobutene such as parleam; synthetic esters and ethers, especially of fatty acids, for instance purcellin oil, isopropyl myristate, 2-ethyl-hexyl palmitate, 2-octyldodecyl stearate, 2-octyl-dodecyl erucate, isostearyl isostearate; hydroxylated esters, for instance isostearyl lactate, octyl hydroxy-stearate, octyldodecyl hydroxystearate, diisostearyl malate, triisocetyl citrate, and fatty alcohol heptanoates, octanoates and decanoates; polyol esters, for instance propylene glycol dioctanoate, neopentyl glycol diheptanoate and diethylene glycol dilsononanoate; and pentaerythritol esters; fatty alcohols containing from 12 to 26 carbon atoms, for instance octyldodecanol, 2-butyloctanol, 2-hexyl-decanol, 2-undecylpentadecanol and oleyl alcohol; partially hydrocarbon-based fluoro oils and/or partially silicone-based fluoro oils; silicone oils, for instance volatile or non-volatile, linear or cyclic polymethylsiloxanes (PDMSs), which are liquid or pasty at room temperature, for instance cyclomethicones, dimethicones, optionally comprising a phenyl group, for instance phenyl trimethicones, phenyltrimethylsiloxy-diphenylsiloxanes, diphenylmethyldimethyltrisiloxanes, diphenyl dimethicones, phenyl dimethicones and polymethylphenylsiloxanes; mixtures thereof.

These oils may be present in a content ranging from 0.01% to 90% and better still from 0.1% to 85% by weight relative to the total weight of the composition.

The composition according to the invention may also comprise one or more physiologically acceptable organic solvents.

These solvents may be generally present in a content ranging from 0.1% to 90%, preferably from 0.5% to 85%, more preferably from 10% to 80% and better still from 30% to 50% by weight, relative to the total weight of the composition.

Mention may be made especially, besides the hydrophilic organic solvents mentioned above, of ketones that are liquid at room temperature such as methyl ethyl ketone, methyl isobutyl ketone, diisobutyl ketone, isophorone, cyclohexanone and acetone; propylene glycol ethers that are liquid at room temperature, such as propylene glycol monomethyl ether, propylene glycol monomethyl ether acetate, and dipropylene glycol mono-n-butyl ether; short-chain esters (containing from 3 to 8 carbon atoms in total), such as ethyl acetate, methyl acetate, propyl acetate, n-butyl acetate and isopentyl acetate; ethers that are liquid at 25° C., such as diethyl ether, dimethyl ether or dichlorodiethyl ether; alkanes that are liquid at 25° C., such as decane, heptane, dodecane, isododecane and cyclohexane; aromatic cyclic compounds that are liquid at 25° C., such as toluene and xylene; aldehydes that are liquid at 25° C., such as benzaldehyde and acetaldehyde, and mixtures thereof.

For the purposes of the present invention, the term “wax” means a lipophilic compound that is solid at room temperature (25° C.), which undergoes a reversible solid/liquid change of state, and which has a melting point of greater than or equal to 25° C., which may be up to 120° C. By bringing the wax to the liquid state (melting), it is possible to make it miscible with the oils possibly present and to form a microscopically homogeneous mixture, but, on returning the temperature of the mixture to room temperature, recrystallization of the wax is obtained in the oils of the mixture. The melting point of the wax may be measured using a differential scanning calorimeter (DSC), for example the calorimeter sold under the name DSC 30 by the company Mettler.

The waxes may be hydrocarbon-based waxes, fluoro waxes and/or silicone waxes and may be of plant, mineral, animal and/or synthetic origin. In particular, the waxes have a melting point of greater than 30° C. and better still greater than 45° C. As waxes that may be used in the composition of the invention, mention may be made of beeswax, carnauba wax or candellila wax, paraffin, microcrystalline waxes, ceresin or ozokerite, synthetic waxes, for instance polyethylene waxes or Fischer-Tropsch waxes, and silicone waxes, for instance alkyl or alkoxy dimethicones containing from 16 to 45 carbon atoms.

The gums are generally polydimethylsiloxanes (PDMSs) of high molecular weight or cellulose gums or polysaccharides, and the pasty substances are generally hydrocarbon-based compounds, for instance lanolins and derivatives thereof, or PDMSs.

The nature and amount of the solid substances depend on the desired mechanical properties and textures. As a guide, the composition may contain from 0.1% to 50% by weight and better still from 1% to 30% by weight of waxes relative to the total weight of the composition.

The composition according to the invention may also comprise, in a particulate phase, pigments and/or nacres and/or fillers usually used in cosmetic compositions.

The composition may also comprise other dyestuffs chosen from water-soluble dyes and/or liposoluble dyes that are well known to those skilled in the art.

The term “pigments” should be understood as meaning white or colored, mineral or organic particles of any shape, which are insoluble in the physiological medium and which are intended to color the composition.

The term “fillers” should be understood as meaning colorless or white, mineral or synthetic, lamellar or non-lamellar particles intended to give body or rigidity to the composition, and/or softness, a matt effect and uniformity to the makeup result.

The term “nacres” should be understood as meaning iridescent particles of any form, produced especially by certain molluscs in their shell, or else synthesized.

The pigments may be present in the composition in a proportion of from 0.01% to 25% and preferably in a proportion of from 3% to 10% by weight of the final composition. They may be white or colored, and mineral or organic. Mention may be made of titanium oxide, zirconium oxide or cerium oxide, and also zinc oxide, iron oxide or chromium oxide, ferric blue, chromium hydrate, carbon black, ultramarines (aluminosilicate polysulfides), manganese pyrophosphate and certain metallic powders such as silver or aluminum powder. Mention may also be made of the D&C pigments and lakes commonly used to give the lips and the skin a makeup effect, which are calcium, barium, aluminum, strontium or zirconium salts.

The nacres may be present in the composition in a proportion of from 0.01% to 20% by weight and preferably in a proportion of about from 3% to 10% by weight. Among the nacres that may be envisaged, mention may be made of natural mother-of-pearl, mica coated with titanium oxide, with iron oxide, with natural pigment or with bismuth oxychloride, and also colored titanium mica.

Among the liposoluble or water-soluble dyes that may be present in the composition, alone or as a mixture, in a proportion of from 0.001% to 15% by weight, preferably 0.01% to 5% by weight and especially from 0.1% to 2% by weight, relative to the total weight of the composition, mention may be made of the disodium salt of ponceau, the disodium salt of alizarin green, quinoline yellow, the trisodium salt of amaranth, the disodium salt of tartrazine, the monosodium salt of rhodamine, the disodium salt of fuchsin, xanthophyll, methylene blue, cochineal carmine, halo-acid dyes, azo dyes, anthraquinone dyes, copper sulfate, iron sulfate, Sudan brown, Sudan red and annatto, and also beetroot juice and carotene.

The composition according to the invention may also comprise one or more fillers, especially in a content ranging from 0.01% to 50% by weight and preferably ranging from 0.02% to 30% by weight, relative to the total weight of the composition. The fillers may be mineral or organic in any form, platelet-shaped, spherical or oblong. Mention may be made of talc, mica, silica, kaolin, polyamide (Nylon®) powder, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie), of acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate or magnesium myristate.

The composition may also comprise an additional polymer such as a film-forming polymer. According to the present invention, the term “film-forming polymer” means a polymer capable, by itself or in the presence of an auxiliary film-forming agent, of forming a continuous film that adheres to a support and especially to keratin materials. Among the film-forming polymers that may be used in the composition of the present invention, mention may be made of synthetic polymers, of free-radical type or of polycondensate type, polymers of natural origin and mixtures thereof, in particular acrylic polymers, polyurethanes, polyesters, polyamides, polyureas and cellulose-based polymers, for instance nitrocellulose.

The composition according to the invention may also comprise ingredients commonly used in cosmetics, such as vitamins, thickeners, gelling agents, trace elements, softeners, sequestrants, fragrances, acidifying or basifying agents, preserving agents, sunscreens, surfactants, antioxidants, hair-loss counteractants, antidandruff agents, propellants and ceramides, or mixtures thereof. Needless to say, a person skilled in the art will take care to select this or these optional additional compound(s), and/or the amount thereof, such that the advantageous properties of the composition according to the invention are not, or are not substantially, adversely affected by the envisaged addition.

The composition according to the invention may be in the form of a suspension, a dispersion, especially of oil in water by means of vesicles; an optionally thickened or even gelled oily solution; an oil-in-water, water-in-oil or multiple emulsion; a gel or a mousse; an oily or emulsified gel; a dispersion of vesicles, especially of lipid vesicles; a two-phase or multiphase lotion; a spray; a loose, compact or cast powder; an anhydrous paste. This composition may have the appearance of a lotion, a cream, a pomade, a soft paste, an ointment, a cast or molded solid especially as a stick or in a dish, or alternatively a compacted solid.

A person skilled in the art will be able to choose the appropriate galenical form, and also the method for preparing it, on the basis of his general knowledge, taking into account firstly the nature of the constituents used, especially their solubility in the support, and secondly the intended application of the composition.

The cosmetic composition according to the invention may be in the form of a care and/or makeup product for bodily or facial skin, the lips, the nails, the eyelashes, the eyebrows and/or the hair, an antisun or self-tanning product, or a hair product for caring for, treating, shaping, making up or dyeing the hair.

It may also be in the form of a makeup composition, especially a complexion product such as a foundation, a makeup rouge or an eyeshadow; a lip product such as a lipstick or a lipcare product; a concealer product; a blusher, a mascara or an eyeliner; an eyebrow makeup product, a lip pencil or an eye pencil; a nail product such as a nail varnish or a nailcare product; a body makeup product; a hair makeup product (hair mascara or hair lacquer).

It may also be in the form of a composition for protecting or caring for the skin of the face, the neck, the hands or the body, especially an antiwrinkle composition or a moisturizing or treating composition; an antisun composition or artificial tanning composition.

It may also be in the form of a hair product, especially for dyeing, holding the hairstyle, shaping the hair, caring for, treating or cleansing the hair, such as shampoos, hairsetting gels or lotions, blow-drying lotions, and fixing and styling compositions such as lacquers or sprays.

A subject of the invention is also a cosmetic process for making up or caring for keratin materials, especially bodily or facial skin, the lips, the nails, the eyelashes, the eyebrows and/or the hair, comprising the application to said materials of a cosmetic composition as defined above.

The invention is illustrated in greater detail in the examples that follow.

### Method for Measuring the Wavelength (Emission and Absorption

The wavelength measurement is performed using a Varian Cary Eclipse fluorimeter.

Unless otherwise mentioned, this measurement is performed in the following manner: 20 mg of product are placed in a 50 ml cylinder. To dissolve the product, said cylinder is filled to 50 ml with a suitable solvent, for example dichloromethane (DCM), chloroform or dimethyl sulfoxide (DMSO). The resulting solution is mixed and 250 microliters are taken and placed in a 50 ml cylinder, and the volume is then made up to 50 ml again with the solvent.

The whole is mixed and a sample of the solution is taken and placed in a closed quartz cell 10 mm thick, which is then placed in the measuring chamber.

### EXAMPLE 1

### 1. First step

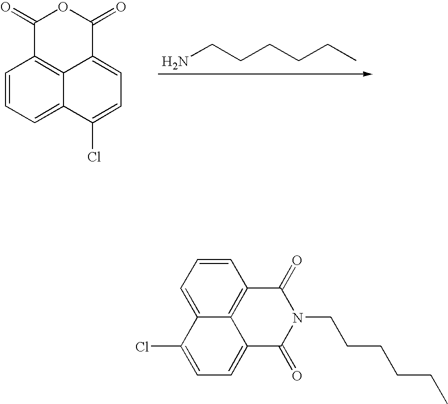

55.4 g (0.23 mol) of 4-chloro-1,8-naphthalic anhydride are placed in a 2 liter round-bottomed flask, under an inert atmosphere (nitrogen), and 750 ml of toluene are then added. The mixture is stirred at 500 rpm for a few minutes, then heated to 90° C. and 24.6 g (0.24 mol) of penta-1-olamine predissolved in 150 ml of toluene are then added dropwise. The mixture is heated to reflux and a further 50 ml of NMP are then added. Refluxing is continued for 16 hours. The resulting reaction mixture is then allowed to cool to room temperature. The product is concentrated under reduced pressure and is precipitated. The precipitate is washed twice with 75 ml of dilute HCl and then with 200-ml of water. The organic phase is recovered and dried under reduced pressure. 69.3 g of product are obtained (91.7% yield).

### Characterization

¹H NMR (CDCl₃, 400 MHz) δ: 8.63-8.61 (1H), 8.57-8.55 (1H), 8.47-8.45 (1H), 7.84-7.83 (2H), 4.19-4.15 (2H), 3.67-3.64 (2H), 1.79-1.64 (5H), 1.75-1.49 (2H),

### 2. Second Step

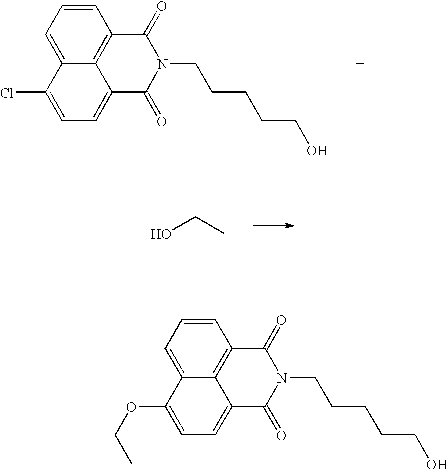

150 ml of ethanol are placed in a one liter three-necked round-bottomed flask, under an inert atmosphere of argon; 2.6 g of sodium hydride (NaH) are added and the mixture is stirred at room temperature for 30 minutes. 19.2 g (0.061 mol) of N-(5-pentanol)-4-chloro-1,8-naphthalimide premixed with 150 ml of ethanol are added and the mixture is stirred vigorously. The mixture is heated at 50° C. for 16 hours, and 30 ml of water are then added. The solvent is evaporated off under reduced pressure to obtain a residue, which is dissolved in dichloromethane. The resulting solution is washed with sodium chloride solution and then with water, dried over sodium sulfate and filtered; the organic phase is evaporated under reduced pressure to give 19.3 g of yellow crystals (97.1% yield).

### Characterization

¹H NMR (CDCl₃, 400 MHz) δ: 8.54-8.47 (3H), 7.67-7.65 (1H), 6.98-6.96 (1H), 4.34-4.29 (2H), 4.17-4.14 (2H), 3.66-3.65 (2H), 1.78-1.72 (3H), 1.67-1.57 (5H), 1.51-1.47 (2H).

### 3. Third Step

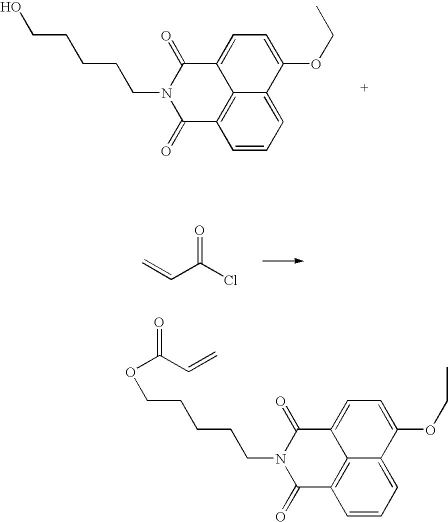

18.0 g (54.8 mmol) of 4-ethoxy-N-(5-pentanol)-1,8-naphthalimide are placed in a three-necked round-bottomed flask equipped with a condenser and placed under an inert atmosphere of argon. 150 ml of dichloromethane are added and the solution is stirred until homogeneous. 26.1 g (187.5 mmol) of triethanolamine are then added, followed by addition of 5.7 g (62.5 mmol) of acryloyl chloride in 20 ml of dichloromethane, with stirring at 15° C. The reaction progress is monitored by TLC (thin-layer chromatography), and when there are no more starting compounds remaining (around 16 hours), 30 ml of water are added. The reaction solution is then evaporated to dryness and the residue is taken up in dichloromethane. The organic phase is washed with saturated sodium bicarbonate solution and then with water and dried over sodium sulfate. The solvents are evaporated off under reduced pressure to give 20.2 g of a pale yellow powder (96.6% yield).

### Characterization

¹H NMR (CDCl₃, 400 MHz) δ: 8.56-8.53 (2H), 8.53-8.51 (1H), 7.70-7.69 (1H), 7.02-7.00 (1H), 6.40-6.35 (1H), 6.16-6.06 (1H), 5.79-5.77 (1H), 4.36-4.31 (2H), 4.19-4.17 (4H), 1.79-1.71 (4H), 1.62-1.60 (3H), 1.58-1.55 (2H).
λₘₐₓ absorption: 406 nm
λₘₐₓ emission: 432 nm

### EXAMPLE 2

### 1. First Step

Similar to the first step of example 1

### 2. Second Step

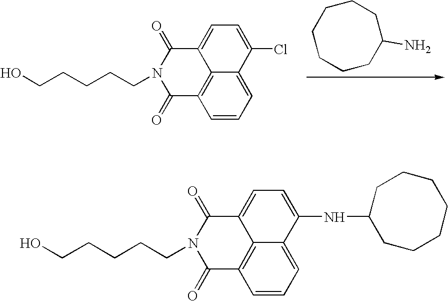

15.0 g (0.047 mol) of 4-chloro-N-(5-pentanol)-1,8-naphthalimide are placed in a 1 liter three-necked round-bottomed flask, under an inert atmosphere (argon), and 50 ml (46.4 g, 0.365 mol) of cyclooctamine are then added. The mixture is heated to 140° C. and stirred until the solution becomes homogeneous. It is then left to react for 18 hours. The resulting reaction mixture is then cooled to room temperature and the residue cyclooctylamine is removed by distillation under reduced pressure. The residue is taken up in 175 ml of dichloromethane and washed with dilute HCl solution, with water and then with sodium bicarbonate solution. The organic phase is dried over sodium sulfate, filtered and then dried under reduced pressure.

17.4 g of an orange-yellow powder are obtained (90.7% yield).

### Characterization

¹H NMR (CDCl₃, 400 MHz) δ: 8.57-8.55 (1H), 8.45-8.43 (1H), 8.06-8.04 (1H), 7.61-7.57 (1H), 6.66-6.64 (1H), 5.23-5.21 (1H), 4.20-4.17 (2H), 3.84-3.82 (1H), 3.67-3.65 (2H), 2.06-2.00 (2H), 1.85-1.45 (19H).

### 3. Third step

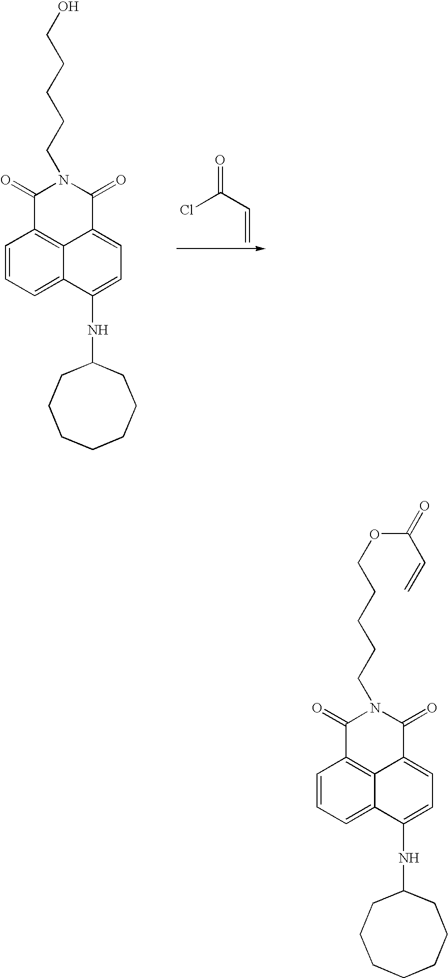

19.0 g (0.046 mol) of N-(5-pentanol)-4-aminocyclooctyl-1,8-naphthalimide are placed in a 1 liter round-bottomed flask, under an inert atmosphere (argon), and 150 ml of dichloromethane (DCM) are then added. The mixture is stirred until a homogeneous solution is obtained. 15.6 g (0.154 mol) of triethanolamine are then added. A mixture of 4.2 g (0.049 mol) of acryloyl chloride in 20 ml of DCM is added dropwise, with stirring (500 rpm) and at 25° C. A further 80 ml of DCM are added. The mixture is left to react for 20 hours and 50 ml of water are then added. The organic phase is washed with water and with sodium bicarbonate, and then again with water. The organic phase is dried over sodium sulfate and filtered. The organic phase is evaporated and 21.5 g of an orange-yellow product are recovered (yield: quantitative).

### Characterization

¹H NMR (CDCl₃, 400 MHz) δ ppm: 8.56-8.54 (1H), 8.45-8.42 (1H), 8.07-8.05 (1H), 7.60-7.58 (1H), 6.65-6.63 (1H), 6.39-6.34 (1H), 6.13-6.06 (1H), 5.80-5.77 (1H), 5.29-5.24 (1H), 4.18-4.13 (4H), 3.84-3.82 (1H), 2.02-1.99 (2H), 1.80-1.48 (18H).
absorption wavelength λ_{absorption}: 368 nm
emission wavelength λmax ₑₘᵢₛₛᵢₒₙ: 508 nm (orange) (solvent: DCM)

### EXAMPLE 3

A homopolymer is prepared from a monomer according to the invention

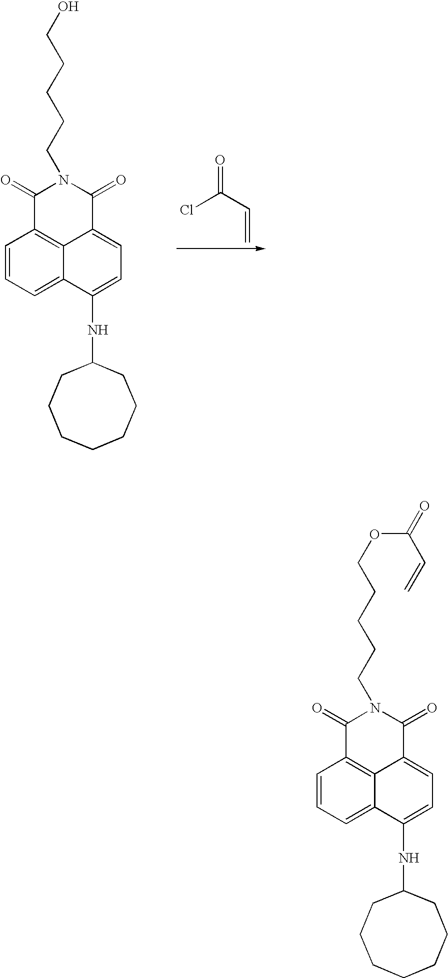

1.0 g (2.6 mmol) of monomer prepared in example 1 are dissolved in 10 ml of anhydrous THF, at 60° C. in the presence of Trigonox 21S (100 μL). The mixture is heated to 90° C. and stirring is continued for 20 hours. Since the viscosity of the solution increases, the medium is diluted by adding 20 ml of THF and is then precipitated dropwise from 500 ml of acetone cooled to 0° C. The polymer is then dried in an oven (50° C.) under vacuum.

0.43 g of polymer are obtained, i.e. a yield of 43%.
absorption wavelength λ_{absorption}: 404 nm
emission wavelength λₘₐₓ emission: 464 nm

### EXAMPLE 4

A homopolymer is prepared from a monomer according to the invention.

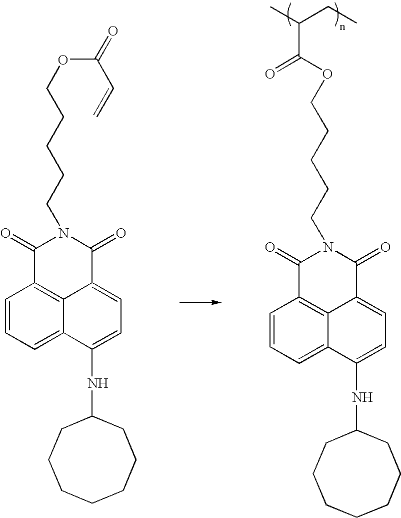

4.0 g (8.6 mmol) of monomer prepared in example 2 are dissolved in 15 ml of THF, at 60° C. in the presence of Trigonox 141 (400 μL). The mixture is heated to 90° C. and stirring is then continued for 20 hours. Since the viscosity of the solution increases, the medium is diluted by adding 20 ml of THF and is then precipitated dropwise from 500 ml of acetone cooled to 0° C. The polymer is then dried in an oven (50° C.) under vacuum. 1.8 g of polymer are obtained, i.e. a yield of 45%.
absorption wavelength λ_{absorption}: 488 nm
emission wavelength λₘₐₓ emission: 522 nm

### EXAMPLE 5

A statistical copolymer comprising a monomer according to the invention is prepared.

20 g of toluene in which are dissolved 5 g of monomer according to example 1 are placed in a reactor, under argon, equipped with a condenser and a stirrer, followed by addition of 35.0 g of isobornyl acrylate, 10.0 g of ethylhexyl acrylate, 30 g of isododecane and 0.6 g of Trigonox 21S (t-butyl peroxy-2-ethylhexanoate) from Akzo Nobel.

The reaction mixture is then heated to 90° C.; the stirring and heating are continued for 4 hours and the mixture is then cooled to room temperature. The resulting polymer is purified by precipitation.

100 g of statistical polymer comprising (weight %): 70% isobornyl acrylate, 20% ethylhexyl acrylate and 10% monomer according to the invention is obtained.
absorption wavelength λ_{absorption}: 448 nm
emission wavelength λₘₐₓ emission: 504 nm

### EXAMPLE 6

A statistical copolymer comprising a monomer according, to the invention is prepared.

20 g of isododecane are placed in a reactor, under argon, equipped with a condenser and a stirrer, followed by addition of 27 g of methyl methacrylate, 17 g of methyl acrylate and 5 g of acrylic acid. The mixture is stirred and a mixture consisting of 1 g of the monomer of example 2 in 20.0 g of toluene is added.

0.5 g of Trigonox 21S (tert-butyl peroxy-2-ethyl-hexanoate) is added and the reaction mixture is then heated to 90° C.; the stirring and heating are continued for 6 hours and the mixture is then cooled to room temperature. The resulting polymer is purified by precipitation.

A statistical polymer comprising (weight %): 54% methyl methacrylate, 34% methyl acrylate, 10% acrylic acid and 2% monomer according to the invention is obtained.

### EXAMPLE 7

An anhydrous foundation is prepared, comprising (weight %):

| | |
|---|---|
| polyethylene wax | 12% |
| volatile silicone oils | 25% |
| phenyl trimethicone | 20% |
| polymethyl methacrylate microspheres | 12% |
| polymer of example 6 | 6% AM |
| isododecane | qs 100% |

(AM: active material)

Preparation: The waxes are melted and, when the whole is clear, the phenyl trimethicone and the silicone oils are added with stirring; the microspheres, the isododecane and the polymer are then added. The mixture is homogenized for 15 minutes and the resulting composition is cast and allowed to cool. An anhydrous foundation is obtained.

### EXAMPLE 8

A statistical copolymer comprising a monomer according to the invention is prepared.

20 g of isododecane are placed in a reactor, under argon, equipped with a condenser and a stirrer, followed by addition of 27 g of methyl methacrylate, 17 g of methyl acrylate and 5 g of acrylic acid. The mixture is stirred and a mixture consisting of 1 g of monomer of example 1 in 20.0 g of toluene is added.

0.5 g of Trigonox 21S (t-butyl peroxy-2-ethylhexanoate) is added and the reaction mixture is then heated to 90° C.; stirring and heating are continued for 6 hours and the mixture is then cooled to room temperature. The resulting polymer is purified by precipitation.

A statistical polymer comprising (weight %) 54% methyl methacrylate, 34% methyl acrylate, 10% acrylic acid and 2% monomer according to the invention is obtained.

### EXAMPLE 9

A nail varnish is prepared, comprising:
5% by weight of polymer according to example 8
qs 100% of organic solvents.

1. A cosmetic or pharmaceutical composition comprising, in a physiologically acceptable medium, at least one polymer comprising at least one monomeric compound of formula (I): 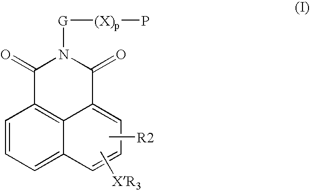
in which:
the groups R2 and X′R3 are present on the same ring or each on a different ring;
R2 and R3 represent, independently of each other, a hydrogen atom, a halogen or a linear, branched and/or is cyclic, saturated and/or unsaturated carbon-based radical containing 1 to 30 carbon atoms; optionally substituted with one or more groups chosen from ═O, OH, NH₂ and halogen atoms; and/or optionally interrupted with one or more heteroatoms chosen from O, N, P, Si and S;
X and X′ represent, independently of each other, —O—, —S—, —SO—, —SO₂—, —NH— and —NR₄— with R₄ representing a linear, branched and/or cyclic, saturated and/or unsaturated carbon-based radical containing 1 to 30 carbon atoms, optionally substituted with one or more groups chosen from ═O, OH, NH₂ and halogen atoms; and/or optionally interrupted with one or more heteroatoms chosen from O, N, P, Si and S;
p is equal to 0 or 1
G is a linear, branched and/or cyclic, saturated and/or unsaturated divalent carbon-based radical containing 1 to 30 carbon atoms, optionally substituted with one or more groups chosen from ═O, OH, NH₂ and halogen atoms; and/or optionally interrupted with one or more heteroatoms chosen from O, N, P, Si and S;
P is a polymerizable group chosen from one of the following formulae: 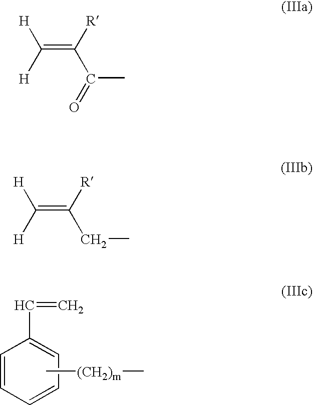
in which:
R′ represents H or a linear or branched, saturated C₁₋₆ hydrocarbon-based radical,
n is equal to 0 or 1 and m is equal to 0 or 1.

2. The composition as claimed in claim 1, in which R2 is a hydrogen atom.

3. The composition as claimed in one of the preceding claims, in which, in the monomeric compound, R3 is a cyclic, linear and/or branched, saturated and/or unsaturated carbon-based and especially hydrocarbon-based radical, optionally comprising a hydrocarbon-based ring that is itself saturated and/or unsaturated, containing 2 to 18, especially 3 to 14 or even 6 to 12 carbon atoms, and may comprise at least one heteroatom, especially one, two or three nitrogen, sulfur and/or oxygen atoms.

4. The composition as claimed in one of the preceding claims, in which, in the monomeric compound, X′R3 is a radical —NH—(CH₂)ₙH; —O—(CH₂)ₙH, for example ethoxy or methoxy; —S—(CH₂)ₙH, —SO—(CH₂)ₙH or —SO₂—(CH₂)ₙH with n being an integer between 1 and 30 and especially between 4 and 12; or alternatively C6-C18-NH-cycloalkyl, especially —NH-cyclohexyl, —NH-cyclooctyl, —NH-cyclodecyl or —NH-cyclododecyl; or alternatively C6-C18-S-cycloalkyl, C6-C18-SO-cycloalkyl or C6-C18-SO₂-cycloalkyl; or alternatively a radical chosen from the following:

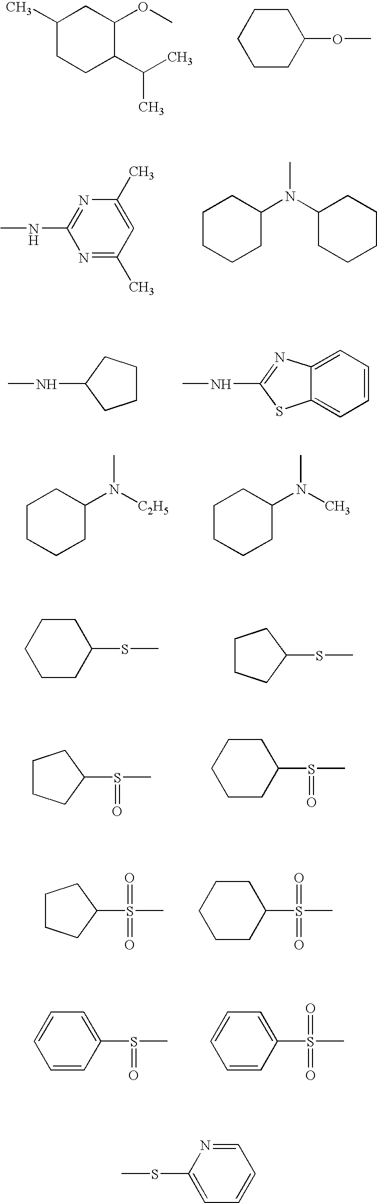

5. The composition as claimed in one of the preceding claims, in which, in the monomeric compound, the divalent radical G is a linear, branched and/or cyclic, saturated or unsaturated divalent hydrocarbon-based radical optionally comprising a hydrocarbon-based ring that is itself saturated or unsaturated, containing in total 2 to 18 and especially 3 to 8 carbon atoms, optionally substituted with one or more groups chosen from ═O, OH, NH₂ and halogen atoms; and/or optionally interrupted with one or more heteroatoms chosen from O, N, P, S and Si.

6. The composition as claimed in one of the preceding claims, in which, in the monomeric compound, G is chosen from linear or branched, saturated divalent hydrocarbon-based radicals optionally comprising a saturated hydrocarbon-based ring, containing in total 2 to 16 and especially 3 to 10 carbon atoms.

7. The composition as claimed in one of the preceding claims, in which, in the monomeric compound, G is chosen from methylene, ethylene, n-propylene, isopropylene (or 1-methylethylene and 2-methyl-ethylene), n-butylene, isobutylene, pentylene, especially n-pentylene, hexylene, especially n-hexylene, or cyclohexylene, heptylene, octylene, cycloctylene, decylene, cyclodecylene, cyclohexyl-dimethylene, dodecylene, cyclododecylene.

8. The composition as claimed in one of the preceding claims, in which, in the monomeric compound, X is chosen from —O—, —S—, —NH— or —NR₄—, preferentially O; and R4 preferentially represents a linear, branched and/or cyclic, saturated or unsaturated hydrocarbon-based radical containing 2 to 12 carbon atoms, optionally substituted with one or more groups chosen from ═O, OH and NH₂.

9. The composition as claimed in one of the preceding claims, in which, in the monomeric compound, the polymerizable group P is chosen from one of the following formulae: 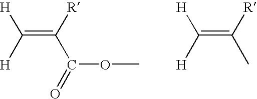
in which R′ represents H or methyl.

10. The composition as claimed in one of the preceding claims, in which the monomeric compound corresponds to one of the following formulae:

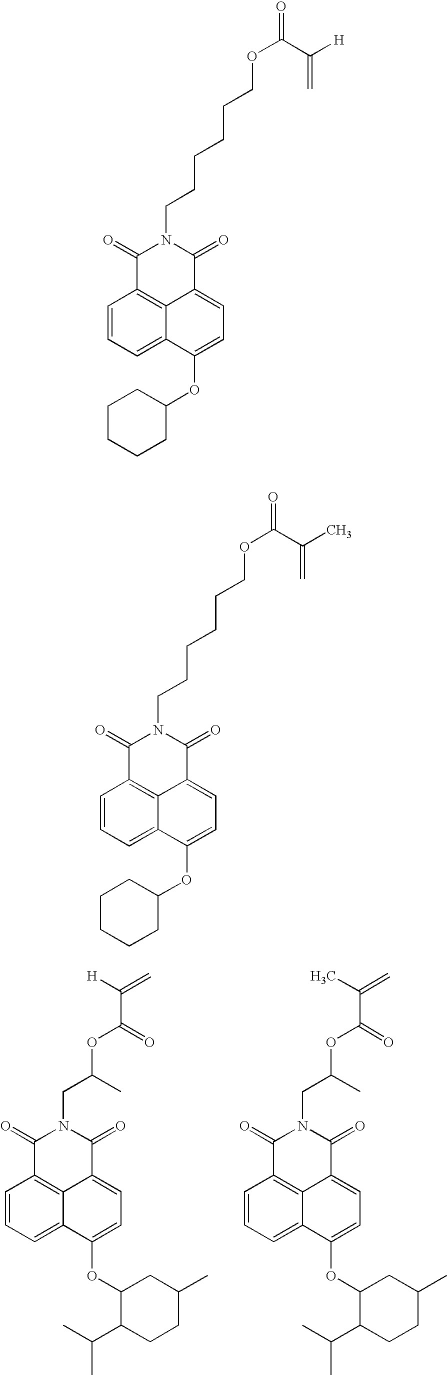

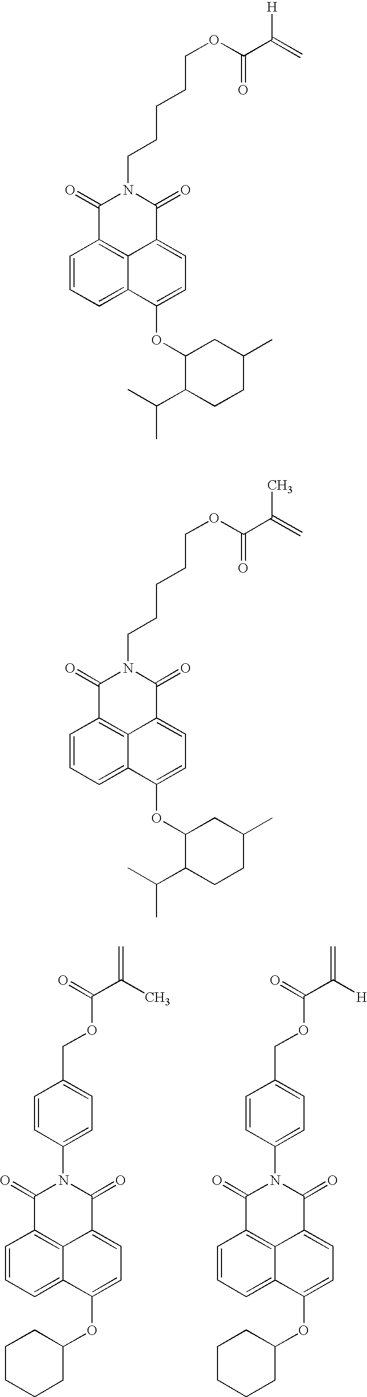

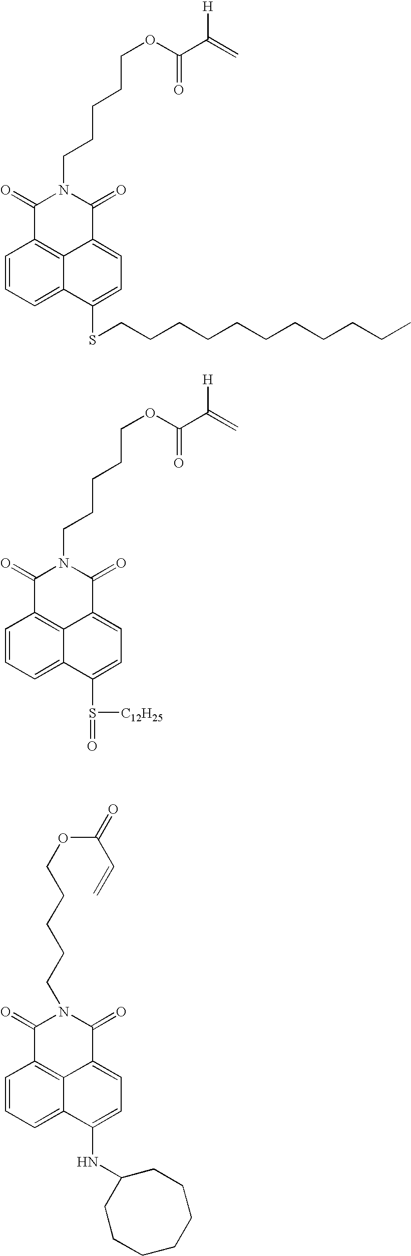

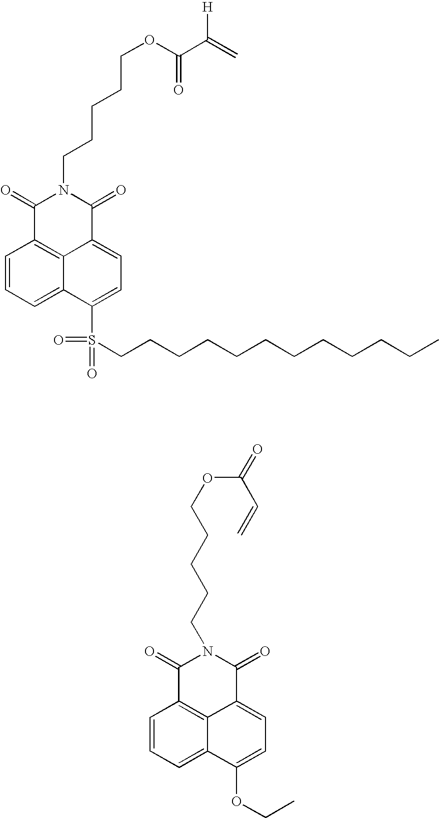

11. The composition as claimed in one of the preceding claims, in which the polymer is a homopolymer of a monomeric compound as defined in one of claims 1 to 10.

12. The composition as claimed in one of claims 1 to 10, in which the polymer is a copolymer comprising only monomeric compounds as defined in one of claims 1 to 10.

13. The composition as claimed in one of claims 1 to 10, in which the polymer is a copolymer comprising at least one monomeric compound as defined in one of claims 1 to 10, and at least one additional comonomer.

14. The composition as claimed in either of claims 12 to 13, in which the polymer is a statistical, alternating, grafted, block or gradient copolymer.

15. The composition as claimed in one of claims 12 to 14, in which the monomeric compound is present in an amount of from 0.01% to 70% by weight relative to the weight of said polymer, especially in an amount of from 0.1% to 50% by weight, in particular from 0.5% to 30% by weight, or even from 1% to 20% by weight and better still from 2% to 10% by weight, the additional comonomers, alone or as a mixture, representing the remainder to 100% by weight.

16. The composition as claimed in one of claims 12 to 15, in which the polymer comprises at least one additional comonomer with an optical effect chosen from the compounds of formula (A), (B) and/or (C): 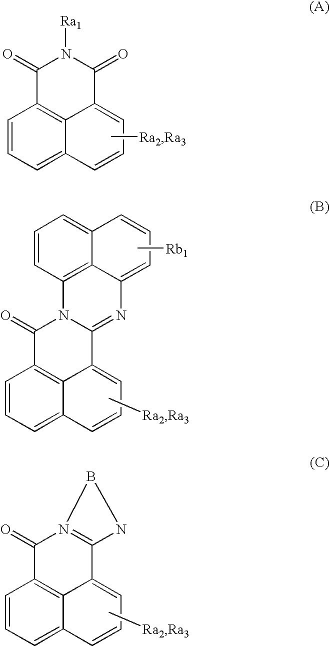
in which:
Ra1 represents a linear, branched and/or cyclic, saturated and/or unsaturated carbon-based radical containing 1 to 32 carbon atoms; optionally substituted with one or more groups chosen from ═O, OH, NH₂ and halogen atoms; and/or optionally interrupted with one or more heteroatoms chosen from O, N, P, Si and S;
Rb1 is chosen from (i) a hydrogen atom, (ii) a halogen, (iii) a linear, branched and/or cyclic, saturated and/or unsaturated carbon-based radical containing 1 to 12 carbon atoms, optionally substituted with one or more groups chosen from ═O, OH and NH₂ and/or optionally interrupted with one or more heteroatoms chosen from O, N, P, Si and S; (iv) a group NRR′ with R and R′ being, independently of each other, a hydrogen atom or a linear, cyclic or branched, saturated C1-6 hydrocarbon-based radical, especially methyl, ethyl, propyl, isopropyl, n-butyl, isobutyl, tert-butyl, pentyl or hexyl;
Ra2 and Ra3, which are present on the same ring or each on a different ring, represent, independently of each other, a hydrogen, a halogen or a group of formula -Xa-Ga—Pa (II), with the proviso that at least one of the radicals Ra2 and/or Ra3 represents a group of formula (II), in which:
Xa is chosen from the groups —O—, —S—, —SO—, —SO₂, —NH— and —NR₄— with R4 representing a linear, branched and/or cyclic, saturated and/or unsaturated carbon-based radical containing 1 to 30 carbon atoms, optionally substituted with one or more groups chosen from ═O, OH, NH₂ and halogen atoms; and/or optionally interrupted with one or more heteroatoms chosen from O, N, P, Si and S;
Ga is a linear, branched and/or cyclic, saturated and/or unsaturated divalent carbon-based radical containing 1 to 32 carbon atoms, optionally substituted with one or more groups chosen from ═O, OH, NH₂ and halogen atoms; and/or optionally interrupted with one or more heteroatoms chosen from O, N, P, Si and S;
Pa is a polymerizable group chosen from one of the following formulae: 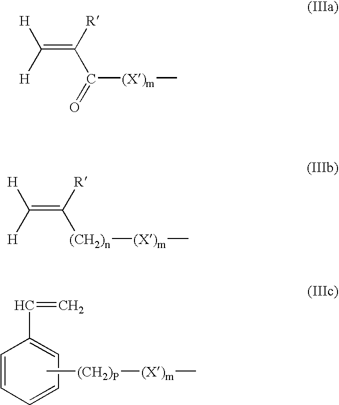
in which:
R′ represents H or a linear or branched, saturated C₁₋₆ hydrocarbon-based radical,
X′ represents O, NH or NR″ with R″ representing a radical chosen from C1-6 alkyl, C6-10 aryl, (C6-10)aryl(C1-6)alkyl and (C1-6)alkyl(C6-10)aryl radicals, the alkyl and/or aryl groups also possibly being substituted with one or more groups chosen from OH, halogen, C1-6 alkoxy and C6-10 aryloxy; and
m is equal to 0 or 1; n is equal to 0 or 1; p is equal to 0, 1 or 2;
B represents one of the following divalent aromatic groups (IVa) to (IVd): 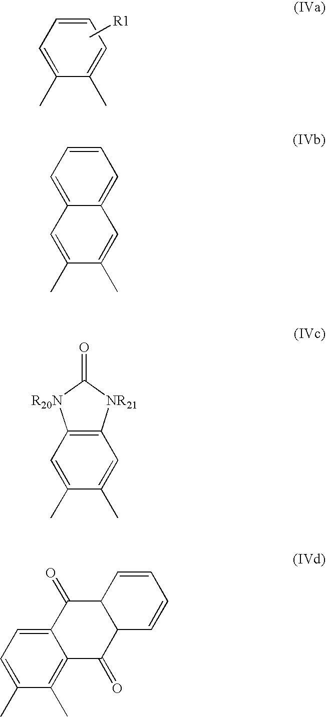
in which:
R1 is a linear, branched and/or cyclic, saturated and/or unsaturated carbon-based radical containing 1 to 32 carbon atoms, optionally substituted with one or more groups chosen from ═O, OH, NH₂ and halogen atoms;
R20 and R21 are, independently of each other, a hydrogen atom, a linear or branched C1-8 alkyl radical or a cyclopentyl, cyclohexyl, cyclooctyl, cyclodecyl, cyclododecyl, benzyl, naphthyl or phenyl radical.

17. The composition as claimed in one of claims 12 to 16, in which the polymer comprises at least one additional comonomer chosen, alone or as a mixture, from the following monomers:
(i) ethylenic hydrocarbons containing from 2 to 10 carbons, such as ethylene, isoprene or butadiene;
(ii) the (meth)acrylates of formula:
CH₂═CHCOOR′₃ or 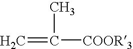
in which R′₃ represents:
a linear or branched alkyl group of 1 to 18 carbon atoms, in which is (are) optionally intercalated one or more heteroatoms chosen from O, N, S and P; said alkyl group also possibly being optionally substituted with one or more substituents chosen from hydroxyl groups, halogen atoms (Cl, Br, I and F), and groups Si(R₄R₅), in which R₄ and R₅, which may be identical or different, represent a C₁ to C₆ alkyl group or a phenyl group;
R′₃ may especially be a methyl, ethyl, propyl, n-butyl, isobutyl, tert-butyl, hexyl, ethylhexyl, octyl, lauryl, isooctyl, isodecyl, dodecyl, cyclohexyl, t-butyl-cyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,
a C₃ to C₁₂ cycloalkyl group such as an isobornyl group,
a C₃ to C₂₀ aryl group such as a phenyl group,
a C₄ to C₃₀ aralkyl group (C₁ to C₈ alkyl group) such as 2-phenylethyl, t-butylbenzyl or benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
said cycloalkyl, aryl, aralkyl, heterocyclic or heterocycloalkyl groups possibly being optionally substituted with one or more substituents chosen from hydroxyl groups, halogen atoms and linear or branched C₁₋₄ alkyl groups in which is (are) optionally intercalated one or more heteroatoms chosen from O, N, S and P, said alkyl groups also possibly being optionally substituted with one or more substituents chosen from hydroxyl groups, halogen atoms (Cl, Br, I and F), and groups Si (R₄R₅), in which R₄ and R₅, which may be identical or different, represent a C₁ to C₆ alkyl group or a phenyl group,
R′₃ may also be a group —(C₂H₄O)ₘ—R″, with m=5 to 150 and R″=H or C₁ to C₃₀ alkyl, for example —POE-methyl or —POE-behenyl;
(iii) the (meth)acrylamides of formula: 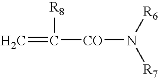
in which R₈ denotes H or methyl; and R₇ and R₆, which may be identical or different, represent:
a hydrogen atom; or
a linear or branched alkyl group of 1 to 18 carbon atoms, in which is (are) optionally intercalated one or more heteroatoms chosen from O, N, S and P; said alkyl group also possibly being optionally substituted with one or more substituents chosen from hydroxyl groups, halogen atoms (Cl, Br, I and F), and groups Si(R₄R₅), in which R₄ and R₅, which may be identical or different, represent a C₁ to C₆ alkyl group or a phenyl group;
R₆ and/or R₇ may especially be a methyl, ethyl, propyl, n-butyl, isobutyl, tert-butyl, hexyl, ethylhexyl, octyl, lauryl, isooctyl, isodecyl, dodecyl, cyclohexyl, t-butylcyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,
a C₃ to C₁₂ cycloalkyl group, such as an isobornyl group,
a C₃ to C₂₀ aryl group such as a phenyl group,
a C₄ to C₃₀ aralkyl group (C₁ to C₈ alkyl group) such as 2-phenylethyl, t-butylbenzyl or benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
said cycloalkyl, aryl, aralkyl, heterocyclic or heterocycloalkyl groups possibly being optionally substituted with one or more substituents chosen from hydroxyl groups, halogen atoms and linear or branched C₁-C₄ alkyl groups in which is (are) optionally intercalated one or more heteroatoms chosen from O, N, S and P, said alkyl groups also possibly being optionally substituted with one or more substituents chosen from hydroxyl groups, halogen atoms (Cl, Br, I and F) and groups Si(R₄R₅), in which R₄ and R₅, which may be identical or different, represent a C₁ to C₆ alkyl group, or a phenyl group;
(iv) the vinyl compounds of formulae:
CH₂═CH—R₉, CH₂═CH—CH₂—R₉ or CH₂═C(CH₃)—CH₂—R₉
in which R₉ is a hydroxyl group, halogen (Cl or F), NH₂, OR₁₀ in which R₁₀ represents a phenyl group or a C₁ to C₁₂ alkyl group (the monomer is a vinyl or allylic ether); acetamide(NHCOCH₃); a group OCOR₁₁ in which R₁₁ represents a linear or branched alkyl group of 2 to 12 carbons (the monomer is a vinyl or allylic ester); or a group chosen from:
a linear or branched alkyl group of 1 to 18 carbon atoms, in which is (are) optionally intercalated one or more heteroatoms chosen from O, N, S and P; said alkyl group also possibly being optionally substituted with one or more substituents chosen from hydroxyl groups, halogen atoms (Cl, Br, I and F) and groups Si(R₄R₅), in which R₄ and R₅, which may be identical or different, represent a C₁ to C₆ alkyl group or a phenyl group;
a C₃ to C₁₂ cycloalkyl group such as isobornyl or cyclohexane,
a C₃ to C₂₀ aryl group such as phenyl,
a C₄ to C₃₀ aralkyl group (C₁ to C₈ alkyl group) such as 2-phenylethyl; benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
said cycloalkyl, aryl, aralkyl, heterocyclic or heterocycloalkyl groups possibly being optionally substituted with one or more substituents chosen from hydroxyl groups, halogen atoms and linear or branched 1 to 4 C alkyl groups in which is (are) optionally intercalated one or more heteroatoms chosen from O, N, S and P, said alkyl groups also possibly being optionally substituted with one or more substituents chosen from hydroxyl groups, halogen atoms (Cl, Br, I and F) and groups Si(R₄R₅) in which R₁ and R₂, which may be identical or different, represent a C₁ to C₆ alkyl group, or a phenyl group;
(v) (meth)acrylic, (meth)acrylamide or vinyl monomers containing a fluoro or perfluoro group, such as ethylperfluorooctyl or 2-ethylperfluorohexyl (meth)acrylate;
(vi) silicone-based (meth)acrylic, (meth)acrylamide or vinyl monomers, such as methacryloxypropyltris(trimethylsiloxy)silane or acryloxypropylpolydimethylsiloxane;
(vii) ethylenically unsaturated monomers comprising at least one carboxylic, phosphoric or sulfonic acid, or anhydride, function, for instance acrylic acid, methacrylic acid, crotonic acid, maleic anhydride, itaconic acid, fumaric acid, maleic acid, acrylamidopropanesulfonic acid, vinylbenzoic acid and vinylphosphoric acid, and the salts thereof;
(viii) ethylenically unsaturated monomers comprising at least one tertiary amine function, for instance 2-vinylpyridine, 4-vinylpyridine, dimethylaminoethyl methacrylate, diethylaminoethyl methacrylate or dimethylaminopropylmethacrylamide, and the salts thereof.

18. The composition as claimed in one of claims 12 to 17, in which the additional comonomer(s) is (are) present in an amount of from 30% to 99.99% by weight, especially in an amount of from 50% to 99.9% by weight, in particular from 70% to 99.5% by weight, or even from 80% to 99% by weight, and better still from 90% to 98% by weight, relative to the weight of the final polymer.

19. The composition as claimed in one of claims 12 to 18, in which the additional comonomers are chosen, alone or as a mixture, from C₁-C₁₈ alkyl or C₃-C₁₂ cycloalkyl(meth)acrylates, and especially from methyl acrylate, methyl methacrylate, isobornyl acrylate, isobornyl methacrylate, isobutyl acrylate, isobutyl methacrylate, 2-ethylhexyl acrylate, 2-ethylhexyl methacrylate, dodecyl acrylate, dodecyl methacrylate, stearyl acrylate, stearyl methacrylate, trifluoroethyl acrylate and trifluoroethyl methacrylate; or alternatively acrylic acid, methacrylic acid, methacryloxypropyltris(trimethylsiloxy)silane, acryl-oxypropyltris(trimethylsiloxy)silane, acryloxypropyl-polydimethylsiloxane and methacryloxypropylpoly-dimethylsiloxane.

20. The composition as claimed in one of the preceding claims, in which the polymer has a weight-average molecular mass (Mw) of between 5000 and 600 000 g/mol, especially between 10 000 and 300 000 g/mol and better still between 20 000 and 150 000 g/mol.

21. The composition as claimed in one of the preceding claims, in which the polymer is present, alone or as a mixture, in an amount of from 0.01% to 60% by weight, preferably 0.1% to 50% by weight, especially 1% to 25% by weight or even 3% to 15% by weight and better still 5% to 12% by weight, relative to the total weight of the composition.

22. The composition as claimed in one of the preceding claims, in which the physiologically acceptable medium comprises a hydrophilic medium comprising water or a water/hydrophilic organic solvent(s) mixture and/or comprises a fatty phase.

23. The composition as claimed in one of the preceding claims, in which the fatty phase comprises waxes, pasty fatty substances, gums, lipophilic organic solvents and oils, and/or mixtures thereof.

24. The composition as claimed in one of the preceding claims, also comprising a particulate phase that may comprise pigments and/or nacres and/or fillers.

25. The composition as claimed in one of the preceding claims, comprising dyestuffs chosen from water-soluble dyes and/or liposoluble dyes.

26. The composition as claimed in one of the preceding claims, comprising at least one additional polymer such as a film-forming polymer.

27. The composition as claimed in one of the preceding claims, comprising at least one ingredient chosen from vitamins, thickeners, gelling agents, trace elements, softeners, sequestrants, fragrances, acidifying or basifying agents, preserving agents, sunscreens, surfactants, antioxidants, hair-loss counteractants, antidandruff agents, propellants and ceramides, or mixtures thereof.

28. The composition as claimed in one of the preceding claims, which is in the form of a suspension, a dispersion especially of oil in water by means of vesicles; an optionally thickened or even gelled oily solution; an oil-in-water, water-in-oil or multiple emulsion; a gel or a mousse; an oily or emulsified gel; a dispersion of vesicles, especially lipid vesicles; a two-phase or multi-phase lotion; a spray; a loose, compact or cast powder; an anhydrous paste; a lotion, a cream, a pomade, a soft paste, an ointment, a cast or molded solid especially as a stick or in a dish, or alternatively a compacted solid.

29. The composition as claimed in one of the preceding claims, which is in the form of a care and/or makeup product for bodily or facial skin, the lips, the nails, the eyelashes, the eyebrows and/or the hair, an antisun or self-tanning product, or a hair product for caring for, treating, shaping, making up or dyeing the hair.

30. The composition as claimed in one of the preceding claims, which is in the form of a makeup composition, especially a complexion product such as a foundation, a makeup rouge or an eyeshadow; a lip product such as a lipstick or a lipcare product; a concealer product; a blusher, a mascara or an eyeliner; an eyebrow makeup product, a lip pencil or an eye pencil; a nail product such as a nail varnish or a nailcare product; a body makeup product; a hair makeup product (hair mascara or hair lacquer); a composition for protecting or caring for the skin of the face, the neck, the hands or the body, especially an antiwrinkle composition or a moisturizing or treating composition; an antisun or artificial tanning composition; a hair product, especially for dyeing, holding the hairstyle, shaping the hair, caring for, treating or cleansing the hair, such as shampoos, hairsetting gels or lotions, blow-drying lotions, and fixing and styling compositions such as lacquers or sprays.

31. A cosmetic process for making up or caring for keratin materials, especially bodily or facial skin, the lips, the nails, the eyelashes, the eyebrows and/or the hair, comprising the application to said materials of a cosmetic composition as defined in any one of claims 1 to 30.

32. A monomeric compound of formula (I): 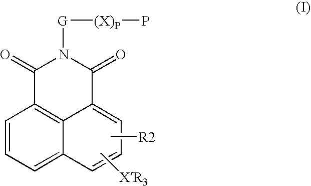
in which:
the groups R2 and X′R3 are present on the same ring or each on a different ring;
R2 and R3 represent, independently of each other, a hydrogen atom, a halogen or a linear, branched and/or cyclic, saturated and/or unsaturated carbon-based radical, containing 1 to 30 carbon atoms; optionally substituted with one or more groups chosen from ═O, OH, NH₂ and halogen atoms; and/or optionally interrupted with one or more heteroatoms chosen from O, N, P, Si and S;
X and X′ represent independently of one another —O—, —S—, —SO—, —SO₂—, —NH— and —NR₄— groups with R₄ representing a linear, branched and/or cyclic, saturated and/or unsaturated carbon-based radical containing 1 to 30 carbon atoms, optionally substituted with one or more groups chosen from ═O, OH, NH₂ and halogen atoms; and/or optionally interrupted with one or more heteroatoms chosen from O, N, P, Si and S;
p is equal to 0 or 1,
G is a linear, branched and/or cyclic, saturated and/or unsaturated divalent carbon-based radical, containing 1 to 30 carbon atoms, optionally substituted with one or more groups chosen from ═O, OH, NH₂ and halogen atoms; and/or optionally interrupted with one or more heteroatoms chosen from O, N, P, Si and S;
P is a polymerizable group chosen from one of the following formulae: 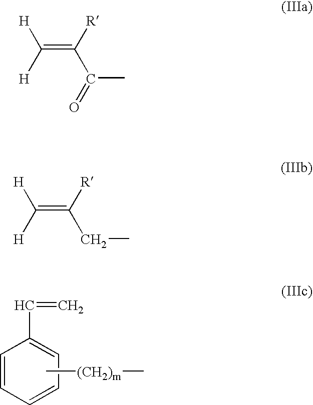
in which:
R′ represents H or a linear or branched, saturated C1-6 hydrocarbon-based radical,
m is equal to 0 or 1;
given that:
when R2=H and simultaneously P is of formula (IIIb), then X′R3 is other than OCH₃;
when R2=H and simultaneously P is of formula (IIIa), and X is equal to O, NH or NR₄, and X′ is equal to O, S or NR₄, then R₃ is chosen from (i) optionally substituted and/or optionally interrupted saturated, linear or branched C2-C5 or C7-C24 alkyl radicals and (ii) optionally substituted and/or optionally interrupted saturated cyclic C5-C18 alkyl radicals.

33. The monomeric compound as claimed in claim 32, in which R2 is a hydrogen atom.

34. The monomeric compound as claimed in either of claims 32 and 33, in which R3 is a cyclic, linear and/or branched, saturated and/or unsaturated carbon-based and especially hydrocarbon-based radical, optionally comprising a hydrocarbon-based ring that is itself saturated and/or unsaturated, containing 2 to 18, especially 3 to 14 or even 6 to 12 carbon atoms, and may comprise at least one heteroatom, especially one, two or three nitrogen, sulfur and/or oxygen atoms.

35. The monomeric compound as claimed in one of claims 32 to 34, in which X′R3 is a radical —NH—(CH₂)ₙH; —O—(CH₂)ₙH, for example ethoxy or methoxy; —S—(CH₂)ₙH, —SO—(CH₂)ₙH or —SO₂—(CH₂)ₙH with n being an integer between 1 and 30 and especially between 4 and 12; or alternatively C6-C18-NH-cycloalkyl, especially —NH-cyclohexyl, —NH-cyclooctyl, —NH-cyclodecyl or —NH-cyclododecyl; or alternatively C6-C18-S-cycloalkyl, C6-C18-SO-cycloalkyl or C6-C18-SO₂-cycloalkyl; or alternatively a radical chosen from the following:

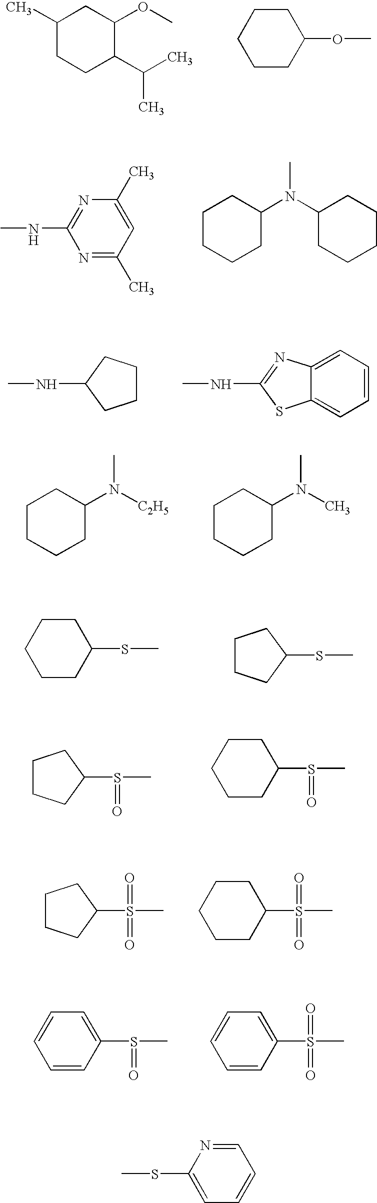

36. The monomeric compound as claimed in one of claims 32 to 35, in which the divalent radical G is a linear, branched and/or cyclic, saturated or unsaturated divalent hydrocarbon-based radical optionally comprising a hydrocarbon-based ring that is itself saturated or unsaturated, containing in total 2 to 18 and especially 3 to 8 carbon atoms, optionally substituted with one or more groups chosen from ═O, OH, NH₂ and halogen atoms; and/or optionally interrupted with one or more heteroatoms chosen from O, N, P, S and Si.

37. The monomeric compound as claimed in one of claims 32 to 36, in which G is chosen from linear or branched, saturated divalent hydrocarbon-based radicals optionally comprising a saturated hydrocarbon-based ring, containing in total 2 to 16 and especially 3 to 10 carbon atoms.

38. The monomeric compound as claimed in one of claims 32 to 37, in which G is chosen from methylene, ethylene, n-propylene, isopropylene (or 1-methylethylene and 2-methylethylene), n-butylene, isobutylene, pentylene, especially n-pentylene, hexylene, especially n-hexylene or cyclohexylene, heptylene, octylene, cyclooctylene, decylene, cyclodecylene, cyclohexyldimethylene, dodecylene, cyclododecylene.

39. The monomeric compound as claimed in one of claims 32 to 38, in which X is chosen from —O—, —S—, —NH— and —NR₄—, preferentially 0; and R4 preferentially represents a linear, branched and/or cyclic, saturated or unsaturated hydrocarbon-based radical containing 2 to 12 carbon atoms, optionally substituted with one or more groups chosen from ═O, OH and NH₂.

40. The monomeric compound as claimed in one of claims 32 to 39, in which the polymerizable group P is of formula: 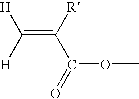
in which R′ represents H or methyl.

41. The monomeric compound as claimed in one of claims 32 to 40, corresponding to formula (I) in which:
R2 is hydrogen, X′ is O, NH or NR₄, and R3 is a linear, branched and/or cyclic, saturated and/or unsaturated carbon-based radical containing 2 to 12 carbon atoms; and/or
G is a linear, branched and/or cyclic, saturated and/or unsaturated divalent carbon-based radical containing 1 to 8 carbon atoms.

42. The monomeric compound as claimed in one of claims 32 to 40, corresponding to one of the following formulae:

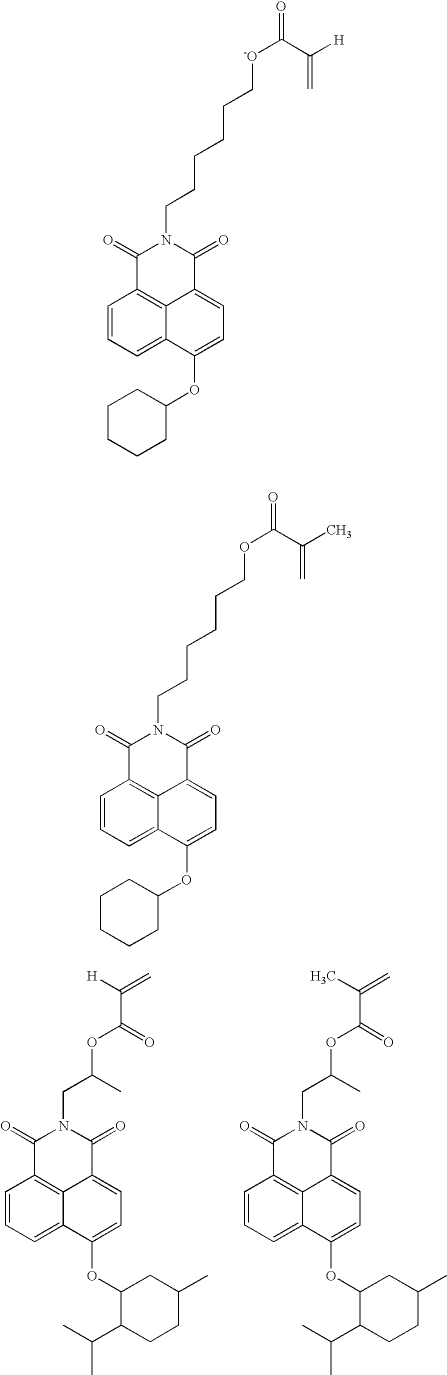

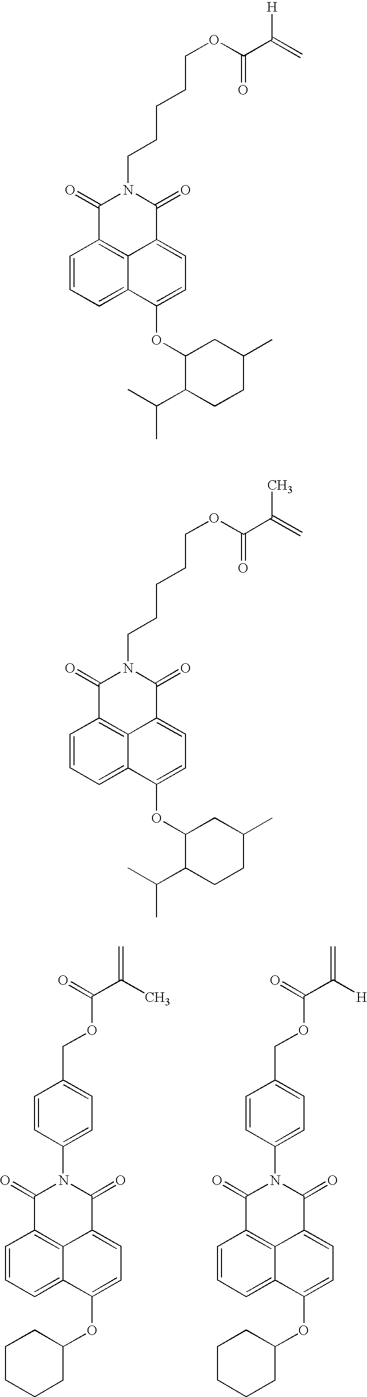

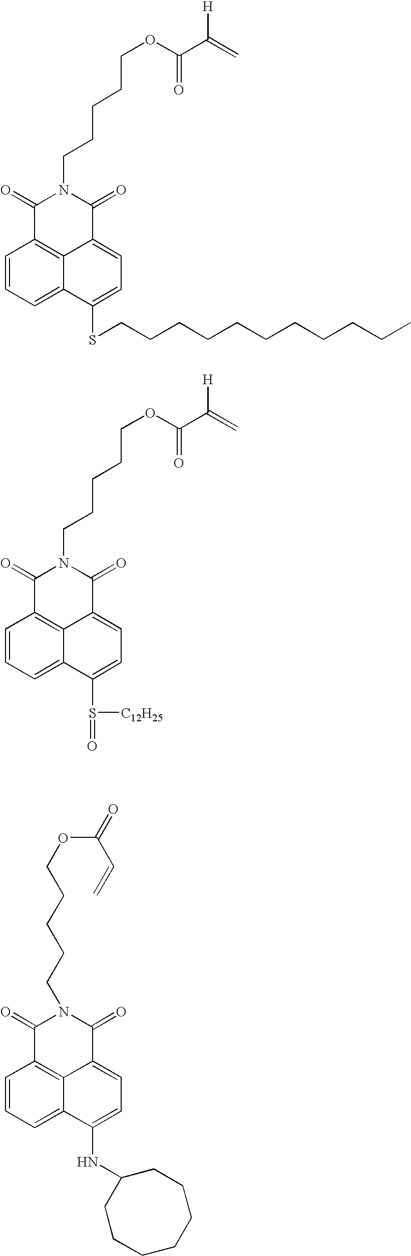

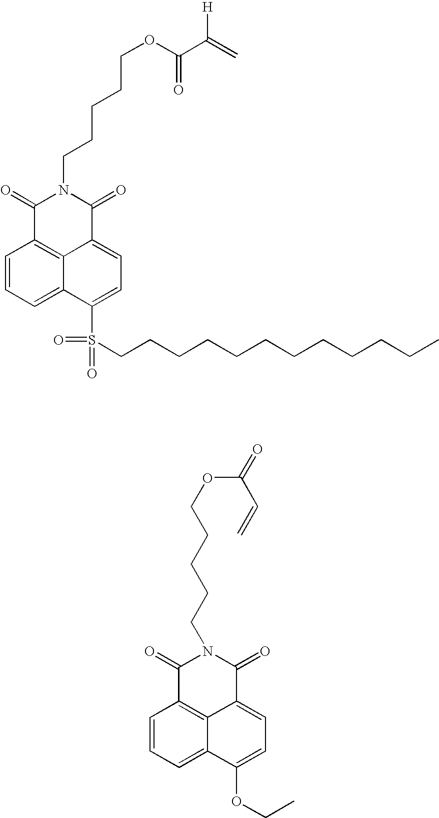

43. A polymer comprising at least one monomeric compound as defined in one of claims 32 to 42.

44. The polymer as claimed in claim 43, characterized in that it is a homopolymer of a monomeric compound as defined in one of claims 32 to 42.

45. The polymer as claimed in claim 43, characterized in that it is a copolymer comprising only monomeric compounds as defined in one of claims 32 to 42.

46. The polymer as claimed in claim 45, characterized in that the monomeric compounds are each present in a proportion of from 0.5% to 99.5% by weight, especially 5% to 95% by weight, or even 10% to 90% by weight and better still each in a proportion of from 30% to 70% by weight, relative to the total weight of the polymer.

47. The polymer as claimed in claim 43, characterized in that it is a copolymer comprising at least one monomeric compound as defined in one of claims 32 to 42, and at least one additional comonomer.

48. The polymer as claimed in claim 47, characterized in that it is a statistical, alternating, grafted, block or gradient copolymer.

49. The polymer as claimed in either of claims 47 and 48, characterized in that the monomeric compound is present in an amount of from 0.01% to 70% by weight relative to the weight of said polymer, especially in an amount of from 0.1% to 50% by weight, in particular from 0.5% to 30% by weight, or even from 1% to 20% by weight and better still from 2% to 10% by weight, the additional comonomers, alone or as a mixture, representing the remainder to 100% by weight.

50. The polymer as claimed in one of claims 47 to 49, characterized in that it comprises at least one additional comonomer with an optical effect chosen from the compounds of formula (A), (B) and/or (C): 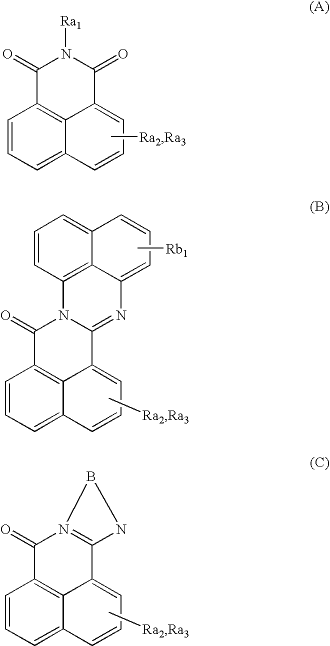
in which:
Ra1 represents a linear, branched and/or cyclic, saturated and/or unsaturated carbon-based radical containing 1 to 32 carbon atoms; optionally substituted with one or more groups chosen from ═O, OH, NH₂ and halogen atoms; and/or optionally interrupted with one or more heteroatoms chosen from O, N, P, Si and S;
Rb1 is chosen from (i) a hydrogen atom, (ii) a halogen, (iii) a linear, branched and/or cyclic, saturated and/or unsaturated carbon-based radical containing 1 to 12 carbon atoms, optionally substituted with one or more groups chosen from ═O, OH and NH₂ and/or optionally interrupted with one or more heteroatoms chosen from O, N, P, Si and S; (iv) a group NRR′ with R and R′ being, independently of each other, a hydrogen atom or a linear, cyclic or branched, saturated C1-6 hydrocarbon-based radical, especially methyl, ethyl, propyl, isopropyl, n-butyl, isobutyl, tert-butyl, pentyl or hexyl;
Ra2 and Ra3, which are present on the same ring or each on a different ring, represent, independently of each other, a hydrogen, a halogen or a group of formula -Xa-Ga—Pa (II), with the proviso that at least one of the radicals Ra2 and/or Ra3 represents a group of formula (II), in which:
Xa is chosen from the groups —O—, —S—, —SO—, —SO₂—, —NH— and —NR₄— with R4 representing a linear, branched and/or cyclic, saturated and/or unsaturated carbon-based radical containing 1 to 30 carbon atoms, optionally substituted with one or more groups chosen from ═O, OH, NH₂ and halogen atoms; and/or optionally interrupted with one or more heteroatoms chosen from O, N, P, Si and S;
Ga is a linear, branched and/or cyclic, saturated and/or unsaturated divalent carbon-based radical containing 1 to 32 carbon atoms, optionally substituted with one or more groups chosen from ═O, OH, NH₂ and halogen atoms; and/or optionally interrupted with one or more heteroatoms chosen from O, N, P, Si and S;
Pa is a polymerizable group chosen from one of the following formulae: 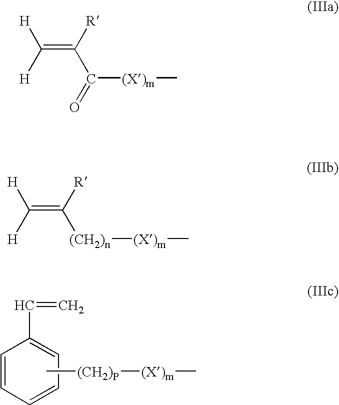
in which:
R′ represents H or a linear or branched, saturated C1-6 hydrocarbon-based radical,
X′ represents O, NH or NR″ with R″ representing a radical chosen from C1-6 alkyl, C6-10 aryl, (C6-10)aryl(C1-6)alkyl and (C1-6)alkyl(C6-10)aryl radicals, the alkyl and/or aryl groups also possibly being substituted with one or more groups chosen from OH, halogen, C1-6 alkoxy and C6-10 aryloxy; and
m is equal to 0 or 1; n is equal to 0 or 1; p is equal to 0, 1 or 2;
B represents one of the following divalent aromatic groups (IVa) to (IVd): 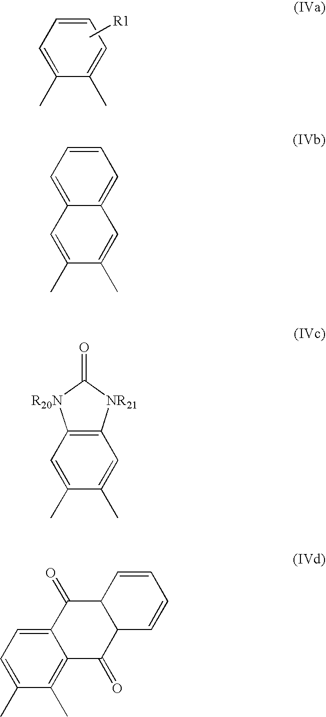
in which:
R1 is a linear, branched and/or cyclic, saturated and/or unsaturated carbon-based radical containing 1 to 32 carbon atoms, optionally substituted with one or more groups chosen from ═O, OH, NH₂ and halogen atoms;
R20 and R21 are, independently of each other, a hydrogen atom, a linear or branched C1-8 alkyl radical or a cyclopentyl, cyclohexyl, cyclooctyl, cyclodecyl, cyclododecyl, benzyl, naphthyl or phenyl radical.

51. The polymer as claimed in one of claims 47 to 50, characterized in that it comprises at least one additional hydrophilic comonomer, or a mixture of such comonomers, which may be present in a proportion of from 1% to 99.99% by weight, especially 2-70% by weight, better still 5-50% by weight or even 10-30% by weight, relative to the total weight of the copolymer.

52. The polymer as claimed in one of claims 47 to 50, characterized in that it comprises at least one additional hydrophobic comonomer, or a mixture of such comonomers, which may be present in a proportion of from 1% to 99.99% by weight, especially 30-98% by weight, better still 50-95% by weight or even 70-90% by weight, relative to the total weight of the copolymer.

53. The polymer as claimed in one of claims 47 to 52, characterized in that it comprises at least one additional comonomer chosen, alone or as a mixture, from the following monomers:
(i) ethylenic hydrocarbons containing from 2 to 10 carbons, such as ethylene, isoprene or butadiene;
(ii) the (meth)acrylates of formula:
CH₂═CHCOOR′₃ or 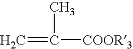
in which R′₃ represents:
a linear or branched alkyl group of 1 to 18 carbon atoms, in which is (are) optionally intercalated one or more heteroatoms chosen from O, N, S and P; said alkyl group also possibly being optionally substituted with one or more substituents chosen from hydroxyl groups, halogen atoms (Cl, Br, I and F), and groups Si (R₄R₅), in which R₄ and R₅, which may be identical or different, represent a C₁ to C₆ alkyl group or a phenyl group;
R′₃ may especially be a methyl, ethyl, propyl, n-butyl, isobutyl, tert-butyl, hexyl, ethylhexyl, octyl, lauryl, isooctyl, isodecyl, dodecyl, cyclohexyl, t-butyl-cyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,
a C₃ to C₁₂ cycloalkyl group such as an isobornyl group,
a C₃ to C₂₀ aryl group such as a phenyl group,
a C₄ to C₃₀ aralkyl group (C₁ to C₈ alkyl group) such as 2-phenylethyl, t-butylbenzyl or benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
said cycloalkyl, aryl, aralkyl, heterocyclic or heterocycloalkyl groups possibly being optionally substituted with one or more substituents chosen from hydroxyl groups, halogen atoms and linear or branched C₁₋₄ alkyl groups in which is (are) optionally intercalated one or more heteroatoms chosen from O, N, S and P, said alkyl groups also possibly being optionally substituted with one or more substituents chosen from hydroxyl groups, halogen atoms (Cl, Br, I and F), and groups Si(R₄R₅), in which R₄ and R₅, which may be identical or different, represent a C₁ to C₆ alkyl group or a phenyl group,
R′₃ may also be a group —(C₂H₄O)ₘ—R″, with m=5 to 150 and R″=H or C₁ to C₃₀ alkyl, for example —POE-methyl or —POE-behenyl;
(iii) the (meth)acrylamides of formula: 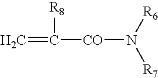
in which R₈ denotes H or methyl; and R₇ and R₆, which may be identical or different, represent:
a hydrogen atom; or
a linear or branched alkyl group of 1 to 18 carbon atoms, in which is (are) optionally intercalated one or more heteroatoms chosen from O, N, S and P; said alkyl group also possibly being optionally substituted with one or more substituents chosen from hydroxyl groups, halogen atoms (Cl, Br, I and F), and groups Si(R₄R₅), in which R₄ and R₅, which may be identical or different, represent a C₁ to C₆ alkyl group or a phenyl group;
R₆ and/or R₇ may especially be a methyl, ethyl, propyl, n-butyl, isobutyl, tert-butyl, hexyl, ethylhexyl, octyl, lauryl, isooctyl, isodecyl, dodecyl, cyclohexyl, t-butylcyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,
a C₃ to C₁₂ cycloalkyl group, such as an isobornyl group,
a C₃ to C₂₀ aryl group such as a phenyl group,
a C₄ to C₃₀ aralkyl group (C₁ to C₈ alkyl group) such as 2-phenylethyl, t-butylbenzyl or benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
said cycloalkyl, aryl, aralkyl, heterocyclic or heterocycloalkyl groups possibly being optionally substituted with one or more substituents chosen from hydroxyl groups, halogen atoms and linear or branched C₁-C₄ alkyl groups in which is (are) optionally intercalated one or more heteroatoms chosen from O, N, S and P, said alkyl groups also possibly being optionally substituted with one or more substituents chosen from hydroxyl groups, halogen atoms (Cl, Br, I and F) and groups Si (R₄R₅), in which R₄ and R₅, which may be identical or different, represent a C₁ to C₆ alkyl group, or a phenyl group;
(iv) the vinyl compounds of formulae:
CH₂═CH—R₉, CH₂═CH—CH₂—R₉ or CH₂═C(CH₃)—CH₂—R₉
in which R₉ is a hydroxyl group, halogen (Cl or F), NH₂, OR₁₀ in which R₁₀ represents a phenyl group or a C₁ to C₁₂ alkyl group (the monomer is a vinyl or allylic ether); acetamide(NHCOCH₃); a group OCOR₁₁ in which R₁₁ represents a linear or branched alkyl group of 2 to 12 carbons (the monomer is a vinyl or allylic ester); or a group chosen from:
a linear or branched alkyl group of 1 to 18 carbon atoms, in which is (are) optionally intercalated one or more heteroatoms chosen from O, N, S and P; said alkyl group also possibly being optionally substituted with one or more substituents chosen from hydroxyl groups, halogen atoms (Cl, Br, I and F) and groups Si(R₄R₅), in which R₄ and R₅, which may be identical or different, represent a C₁ to C₆ alkyl group or a phenyl group;
a C₃ to C₁₂ cycloalkyl group such as isobornyl or cyclohexane,
a C₃ to C₂₀ aryl group such as phenyl,
a C₄ to C₃₀ aralkyl group (C₁ to C₈ alkyl group) such as 2-phenylethyl; benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
said cycloalkyl, aryl, aralkyl, heterocyclic or heterocycloalkyl groups possibly being optionally substituted with one or more substituents chosen from hydroxyl groups, halogen atoms and linear or branched 1 to 4 C alkyl groups in which is (are) optionally intercalated one or more heteroatoms chosen from O, N, S and P, said alkyl groups also possibly being optionally substituted with one or more substituents chosen from hydroxyl groups, halogen atoms (Cl, Br, I and F) and groups Si(R₄R₅) in which R₁ and R₂, which may be identical or different, represent a C₁ to C₆ alkyl group, or a phenyl group;
(v) (meth)acrylic, (meth)acrylamide or vinyl monomers containing a fluoro or perfluoro group, such as ethylperfluorooctyl or 2-ethylperfluorohexyl (meth)acrylate;
(vi) silicone-based (meth)acrylic, (meth)acrylamide or vinyl monomers, such as methacryloxypropyltris(trimethylsiloxy)silane or acryloxypropylpolydimethylsiloxane;
(vii) ethylenically unsaturated monomers comprising at least one carboxylic, phosphoric or sulfonic acid, or anhydride, function, for instance acrylic acid, methacrylic acid, crotonic acid, maleic anhydride, itaconic acid, fumaric acid, maleic acid, acrylamidopropanesulfonic acid, vinylbenzoic acid and vinylphosphoric acid, and the salts thereof;
(viii) ethylenically unsaturated monomers comprising at least one tertiary amine function, for instance 2-vinylpyridine, 4-vinylpyridine, dimethylaminoethyl methacrylate, diethylaminoethyl methacrylate or dimethylaminopropylmethacrylamide, and the salts thereof.

54. The polymer as claimed in claim 53, characterized in that the additional comonomer(s) is (are) present in an amount of from 30% to 99.99% by weight, especially in an amount of from 50% to 99.9% by weight, in particular from 70% to 99.5% by weight or even from 80% to 99% by weight and better still from 90% to 98% by weight relative to the weight of the final polymer.

55. The polymer as claimed in one of claims 47 to 54, characterized in that the additional comonomers are chosen, alone or as a mixture, from C₁-C₁₈ alkyl or C₃-C₁₂ cycloalkyl(meth)acrylates, and especially from methyl acrylate, methyl methacrylate, isobornyl acrylate, isobornyl methacrylate, isobutyl acrylate, isobutyl methacrylate, 2-ethylhexyl acrylate, 2-ethylhexyl methacrylate, dodecyl acrylate, dodecyl methacrylate, stearyl acrylate, stearyl methacrylate, trifluoroethyl acrylate and trifluoroethyl methacrylate; or alternatively acrylic acid, methacrylic acid, methacryloxypropyltris(trimethyl-siloxy)silane, acryloxypropyltris(trimethylsiloxy)-silane, acryloxypropylpolydimethylsiloxane and methacryloxypropylpolydimethylsiloxane.

56. The polymer as claimed in one of claims 47 to 55, characterized in that it has a weight-average molecular mass (Mw) of between 5000 and 600 000 g/mol, especially between 10 000 and 300 000 g/mol and better still between 20 000 and 150 000 g/mol.

57. The use of at least one monomeric compound as defined in one of claims 32 to 42, or of at least one polymer as defined in one of claims 43 to 56, in a composition, for giving said composition optical effects, especially fluorescence or optical-brightening effects.
